SEQUENCE LISTING (54 sequences)

SEQ ID NO: 1 - DNA, 35 bp, artificial sequence
  primer rbc89
ccgtggccacaaacagagactaaagcaagtgttgg

SEQ ID NO: 2 - DNA, 54 bp, artificial sequence
  primer rbc90
tagcggccgcttagtgatggtgatggtgatgcttatccaaaacgtccactgctg

SEQ ID NO: 3 - DNA, 34 bp, artificial sequence
  primer rbc87
gatcatattcactctggaaccgtagtaggtaaac

SEQ ID NO: 4 - DNA, 34 bp, artificial sequence
  primer rbc88
gtttacctactacggttccagagtgaatatgatc

SEQ ID NO: 5 - DNA, 31 bp, artificial sequence
  primer rbc120
tggcggccgccccgggcaacccactagcata

SEQ ID NO: 6 - DNA, 32 bp, artificial sequence
  primer rbc121
ggggatccatggtaaaatcttggtttatttaa

SEQ ID NO: 7 - DNA, 30 bp, artificial sequence
  primer rbc122
taccatggcaccacaaacagagactaaagc

SEQ ID NO: 8 - DNA, 29 bp, artificial sequence
  primer rbc123
tcgaattcttagtgatggtgatggtgatg

SEQ ID NO: 9 - DNA, 26 bp, artificial sequence
  primer rbc124
aagaattcaattaaggaaataaatta

SEQ ID NO: 10 - DNA, 29 bp, artificial sequence
  primer rbc125
ataagcttaattcaatggaagcaatgata

SEQ ID NO: 11 - DNA, 29 bp, artificial sequence
  primer rbc116
gaggtaccaccgccgtatggctgaccggc

SEQ ID NO: 12 - DNA, 30 bp, artificial sequence
  primer rbc117
gcctcgagttgacaattgaatccgattttg

SEQ ID NO: 13 - DNA, 30 bp, artificial sequence
  primer rbc118
gaggtcgacctgcccctatcggaaatagga

SEQ ID NO: 14 - DNA, 29 bp, artificial sequence
  primer rbc119
tcaagcttttcttgatcaatccctttgcc

SEQ ID NO: 15 - DNA, 26 bp, artificial sequence
  primer rbc146
gttacgacttcactccagtcactagc

SEQ ID NO: 16 - DNA, 26 bp, artificial sequence
  primer rbc147
ccgaagagtaactaggaccaatttag

SEQ ID NO: 17 - DNA, 54 bp, artificial sequence; identical to SEQ ID NO: 2
  primer rbc90

SEQ ID NO: 18 - DNA, 23 bp, artificial sequence
  primer ATrbcL
ggtggaggaactttaggacatcc

SEQ ID NO: 19 - DNA, 18 bp, artificial sequence
  primer Trans-Tob-rbcL-1337F
gcgaggcttgcaaatgga

SEQ ID NO: 20 - DNA, 22 bp, artificial sequence
  primer Trans-Tob-rbcL-1449R
gtgatggtgatggtgatgctta

SEQ ID NO: 21 - DNA, 24 bp, artificial sequence
  primer Endo-Tob-rbcL-1289F
ctcgtaatgaaggacgtgatcttg

SEQ ID NO: 22 - DNA, 17 bp, artificial sequence
  primer Endo-Tob-rbcL-1362R
ttccgggctccatttgc

SEQ ID NO: 23 - DNA, 18 bp, artificial sequence
  primer Tobacco 18S-1515F
gtccgcatggcccttatg

SEQ ID NO: 24 - DNA, 22 bp, artificial sequence
  primer Tobacco-18S-1580R
ccttgcttcccattgtaattgc

SEQ ID NO: 25 - DNA, 24 bp, artificial sequence
  primer rbc154
atgcatatggaccagtcatctcgt

SEQ ID NO: 26 - DNA, 29 bp, artificial sequence
  primer rbc155
tagaattcttacgccggaagggcgctgcg

SEQ ID NO: 27 - DNA, 30 bp, artificial sequence
  primer rbc141
gtttgtggtgccatatgggtaaaatcttgg

SEQ ID NO: 28 - DNA, 30 bp, artificial sequence
  primer rbc142
ccaagattttacccatatggcaccacaaac

SEQ ID NO: 29 - DNA, 20 bp, artificial sequence
  primer rbc211
ttctacctccacgcggcatt

SEQ ID NO: 30 - DNA, 22 bp, artificial sequence
  primer rbc212
gcctgattatccctaagcccaa

SEQ ID NO: 31 - DNA, 1771 bp, artificial sequence
  trnV homologous recombination arm from tobacco 16srDNA-trnV
accgccgtatggctgaccggcgattactagcgattccggcttcatgcaggcgagttgcagcctgcaatccgaactgaggacgggtttttggggttagctcaccctcgcgggatcgcgaccctttgtcccggccattgtagcacgtgtgtcgcccagggcataaggggcatgatgacttgacgtcatcctcaccttcctccggcttatcaccggcagtctgttcagggttccaaactcaacgatggcaactaaacacgagggttgcgctcgttgcgggacttaacccaacaccttacggcacgagctgacgacagccatgcaccacctgtgtccgcgttcccgaaggcacccctctctttcaagaggattcgcggcatgtcaagccctggtaaggttcttcgctttgcatcgaattaaaccacatgctccaccgcttgtgcgggcccccgtcaattcctttgagtttcattcttgcgaacgtactccccaggcgggatacttaacgcgttagctacagcactgcacgggtcgatacgcacagcgcctagtatccatcgtttacggctaggactactggggtatctaatcccattcgctcccctagctttcgtctctcagtgtcagtgtcggcccagcagagtgctttcgccgttggtgttctttccgatctctacgcatttcaccgctccaccggaaattccctctgcccctaccgtactccagcttggtagtttccaccgcctgtccagggttgagccctgggatttgacggcggacttaaaaagccacctacagacgctttacgcccaatcattccggataacgcttgcatcctctgtattaccgcggctgctggcacagagttagccgatgcttattccccagataccgtcattgcttcttctccgggaaaagaagttcacgacccgtgggccttctacctccacgcggcattgctccgtcaggctttcgcccattgcggaaaattccccactgctgcctcccgtaggagtctgggccgtgtctcagtcccagtgtggctgatcatcctctcggaccagctactgatcatcgccttggtaagctattgcctcaccaactagctaatcagacgcgagcccctcctcgggcggattcctccttttgctcctcagcctacggggtattagcagccgtttccagctgttgttcccctcccaagggcaggttcttacgcgttactcacccgtccgccactggaaacaccacttcccgtccgacttgcatgtgttaagcatgccgccagcgttcatcctgagccaggatcgaactctccatgagattcatagttgcattacttatagcttccttgttcgtagacaaagcggattcggaattgtctttcattccaaggcataacttgtatccatgcgcttcatattcgcccggagttcgctcccagaaatatagccatccctgccccctcacgtcaatcccacgagcctcttatccattctcattgaacgacggcgggggagcaaatccaactagaaaaactcacattgggcttagggataatcaggctcgaactgatgacttccaccacgtcaaggtgacactctaccgctgagttatatcccttccccgccccatcgagaaatagaactgactaatcctaagtcaaagggtcgagaaactcaacgccactattcttgaacaacttggagccgggccttcttttcgcactattacggatatgaaaataatggtcaaaatcggattcaattgtcaa

SEQ ID NO: 32 - DNA, 1138 bp, artificial sequence
  tobacco chloroplast rps12/7 fragment
ctgcccctatcggaaataggattgactaccgattccgaaggaactggagttacatctcttttccattcaagagttcttatgcgtttccacgcccctttgagaccccgaaaaatggacaaattccttttcttaggaacacatacaagattcgtcactacaaaaaggataatggtaaccctaccattaactacttcatttatgaatttcatagtaatagaaatacatgtcctaccgagacagaatttggaacttgctatcctcttgcctagcaggcaaagatttacctccgtggaaaggatgattcattcggatcgacatgagagtccaactacattgccagaatccatgttgtatatttgaaagaggttgacctccttgcttctctcatggtacactcctcttcccgccgagccccttttctcctcggtccacagagacaaaatgtaggactggtgccaacaattcatcagactcactaagtcgggatcactaactaatactaatctaatataatagaaaatactaatataatagaaaagaactgtcttttctgtatactttccccggttccgttgctaccgcgggctttacgcaatcgatcggattagatagatatcccttcaacataggtcatcgaaaggatctcggagacccaccaaagtacgaaagccaggatctttcagaaaacggattcctattcaaagagtgcataaccgcatggataagctcacactaacccgtcaatttgggatccaaattcgagattttccttgggaggtatcgggaaggattttgaatggaataatatcgattcatacagaagaaaaggttctctattgattcaaacactgtacctaacctatgggatagggatcgaggaaggggaaaaaccgaagatttcacatggtacttttatcaatctgatttatttcgtacctttcgttcaatgagaaaatgggtaaaattctacaggatcaaacctatgggacttaaggaatgatataaaaaaaagagagggaaaatattcatattaaataaatatgaagtagaagaacccagattccaaatgaacaaattcaaacttgaaaaggatcttccttattcttgaagaatgaggggcaaagggattgatcaagaa

SEQ ID NO: 33 - DNA, 1452 bp, artificial sequence
  tobacco rbcL transgene (cpNTrbcL) in pTCP102
atggcaccacaaacagagactaaagcaagtgttggattcaaagctggtgttaaagagtacaaattgacttattatactcctgagtaccaaaccaaggatactgatatattggcagcattccgagtaactcctcaacctggagttccacctgaagaagcaggggccgcggtagctgccgaatcttctactggtacatggacaactgtatggaccgatggacttaccagccttgatcgttacaaagggcgatgctaccgcatcgagcgtgttgttggagaaaaagatcaatatattgcttatgtagcttaccctttagacctttttgaagaaggttctgttaccaacatgtttacttccattgtaggtaacgtatttgggttcaaagccctgcgcgctctacgtctggaagatctgcgaatccctcctgcttatgttaaaactttccaaggtccgcctcatgggatccaagttgaaagagataaattgaacaagtatggtcgtcccctgttgggatgtactattaaacctaaattggggttatctgctaaaaactacggtagagctgtttatgaatgtcttcgcggtggacttgattttaccaaagatgatgagaacgtgaactcacaaccatttatgcgttggagagatcgtttcttattttgtgccgaagcactttataaagcacaggctgaaacaggtgaaatcaaagggcattacttgaatgctactgcaggtacatgcgaagaaatgatcaaaagagctgtatttgctagagaattgggcgttccgatcgtaatgcatgactacttaacggggggattcaccgcaaatactagcttggctcattattgccgagataatggtctacttcttcacatccaccgtgcaatgcatgcggttattgatagacagaagaatcatggtatccacttccgggtattagcaaaagcgttacgtatgtctggtggagatcatattcactctggaaccgtagtaggtaaacttgaaggtgaaagagacataactttgggctttgttgatttactgcgtgatgattttgttgaacaagatcgaagtcgcggtatttatttcactcaagattgggtctctttaccaggtgttctacccgtggcttcaggaggtattcacgtttggcatatgcctgctctgaccgagatctttggggatgattccgtactacagttcggtggaggaactttaggacatccttggggtaatgcgccaggtgccgtagctaatcgagtagctctagaagcatgtgtaaaagctcgtaatgaaggacgtgatcttgctcaggaaggtaatgaaattattcgcgaggcttgcaaatggagcccggaactagctgctgcttgtgaagtatggaaagagatcgtatttaattttgcagcagtggacgttttggataagcatcaccatcaccatcactaa

SEQ ID NO: 34 - protein, 483 aa, artificial sequence
  amino acid sequence of tobacco rbcL transgene (cpNTrbcL) in pTCP102
MAPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYVKTFQGPPHGIQVERDKLNKYGRPLLGCTIKPKLGLSAKNYGRAVYECLRGGLDFTKDDENVNSQPFMRWRDRFLFCAEALYKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVKARNEGRDLAQEGNEIIREACKWSPELAAACEVWKEIVFNFAAVDVLDKHHHHHH

SEQ ID NO: 35 - DNA, 1401 bp, artificial sequence
  R. rubrum rbcM transgene (RRrbcM) in pTCP107
atggaccagtcatctcgttacgtcaatctggcgctcaaggaagaggatctgatcgccggcggcgagcatgtgctttgtgcctatatcatgaagcccaaggccggatatggctatgtggcgaccgcggcgcatttcgccgccgagagttcgacgggcaccaacgtcgaggtctgcaccaccgacgatttcacccggggcgtcgacgccctggtctatgaggtggacgaggcccgcgagctgaccaagatcgcctatccggtggctttgttcgaccgcaacatcaccgacggcaaggcgatgatcgcctcgttcctgacgctcaccatgggaaacaaccagggtatgggcgacgtggaatacgccaagatgcacgatttctatgtgcccgaggcttatcgcgccctgtttgatggcccgagcgtcaatatctcggccctgtggaaagtgctggggcggcccgaggtcgacggcggtctggtcgtcggcacgatcatcaagccgaagctcggcctgcgtcccaagcccttcgccgaggcctgccacgccttctggctgggcggcgacttcatcaagaacgacgagccccagggcaatcagcccttcgcccccttgcgcgacaccatcgccctggtcgccgacgccatgaggcgggcccaggacgagaccggcgaggccaagctgttctcggccaatatcaccgccgacgatcccttcgagatcatcgcccgtggcgagtatgtgctggagaccttcggcgagaacgcctcgcatgtcgccttgctggtcgacggctatgtcgccggcgccgcggcgatcaccacggcgcgccgccgcttccccgataacttcttgcattatcaccgggctggccacggcgccgtcacctcgccccagtccaagcgcggctataccgccttcgtccattgcaagatggcccgcctgcagggcgccagcggcatccacaccggcaccatgggctttggcaagatggaaggcgagtccagcgaccgcgccatcgcctatatgctgacccaggacgaggcccaggggccgttctaccgtcaatcctggggcggcatgaaggcttgtacgccgatcatcagcggcggcatgaacgccctgcgcatgcccggcttcttcgagaacctgggtaatgccaatgtcatcttgaccgccggcggcggcgccttcggccatatcgacggcccggtggccggggcgcggtcgttgcgtcaagcctggcaagcctggcgggacggggttccggttctggactatgcccgcgagcacaaggaactggcccgcgccttcgagtccttccccggcgacgccgaccagatctatccgggctggcgcaaggccctgggcgtcgaggacacccgcagcgcccttccggcgtaa

SEQ ID NO: 36 - protein, 466 aa, artificial sequence
  amino acid sequence of R. rubrum rbcM transgene (RRrbcM) in pTCP107
MDQSSRYVNLALKEEDLIAGGEHVLCAYIMKPKAGYGYVATAAHFAAESSTGTNVEVCTTDDFTRGVDALVYEVDEARELTKIAYPVALFDRNITDGKAMIASFLTLTMGNNQGMGDVEYAKMHDFYVPEAYRALFDGPSVNISALWKVLGRPEVDGGLVVGTIIKPKLGLRPKPFAEACHAFWLGGDFIKNDEPQGNQPFAPLRDTIALVADAMRRAQDETGEAKLFSANITADDPFEIIARGEYVLETFGENASHVALLVDGYVAGAAAITTARRRFPDNFLHYHRAGHGAVTSPQSKRGYTAFVHCKMARLQGASGIHTGTMGFGKMEGESSDRAIAYMLTQDEAQGPFYRQSWGGMKACTPIISGGMNALRMPGFFENLGNANVILTAGGGAFGHIDGPVAGARSLRQAWQAWRDGVPVLDYAREHKELARAFESFPGDADQIYPGWRKALGVEDTRSALPA

SEQ ID NO: 37 - DNA, 792 bp, artificial sequence
  promoter sequence aadA3
atgggtgttgctgttactattgaaatctctaatcaattgtccgaagttttgtctgttatcgaacgtcatcttgaatccactcttctagctgttcatctgtatggtagtgctgttgatggaggattgaaaccttactcagatatcgatctacttgttactgttgctgtgaaacttgatgaaactactcgtagagctctgttgaatgatctaatggaagcaagcgcatttcctggtgaatctgaaacccttcgtgcaatcgaagtaacactagttgtgcacgatgatatcatcccttggagatatccagctaaacgagaactgcaatttggagaatggcaacgcaacgatatcttggccggaatcttcgaacccgctatgatcgatattgatctagcaattcttctaactaaagcccgtgaacattctgttgcgctggtagggcctgctgctgaggaattcttcgatccagtacccgagcaagacttgtttgaagcattacgtgagactcttaaactatggaattctcaaccggattgggctggagacgaacgaaacgtagtattgaccttatccagaatttggtattctgcaattactggtaaaattgctcctaaagatgtagcagccgactgggcgattaaacgacttcctgctcaataccagccagtactattggaggctaaacaagcatatctaggtcagaaagaagatcaccttgcctcacgcgcggaccatcttgaggaattcattcactacgtaaagggtgagattactaaagtagttggaaagtag

SEQ ID NO: 38 - protein, 263 aa, artificial sequence
  amino acid sequence of aadA3
MGVAVTIEISNQLSEVLSVIERHLESTLLAVHLYGSAVDGGLKPYSDIDLLVTVAVKLDETTRRALLNDLMEASAFPGESETLRAIEVTLVVHDDIIPWRYPAKRELQFGEWQRNDILAGIFEPAMIDIDLAILLTKAREHSVALVGPAAEEFFDPVPEQDLFEALRETLKLWNSQPDWAGDERNVVLTLSRIWYSAITGKIAPKDVAADWAIKRLPAQYQPVLLEAKQAYLGQKEDHLASRADHLEEFIHYVKGEITKVVGK

SEQ ID NO: 39 - DNA, 1780 bp, Solanum tuberosum
ttgacaattgaatccgattttgaccattattttcatatccgtaatagtgcgaaaagaaggcccggctccaagttgttcaagaatagtggcgttgagtttctcgaccctttgacttaggattagtcagttctatttctcgatggggcggggaagggatataactcagcggtagagtgtcaccttgacgtggtggaagtcatcagttcgagcctgattatccctaagcccaatgtgagtttttctagttggatttgctcccccgccgtcgttcaatgagaatggataagaggctcgtgggattgacgtgagggggcagggatgactatatttctgggagcgaactccgggcgaatatgaagcgcatggatacaagttatgccttggaatgaaagacaattccgaatccgctttgtctacgaacaaggaagctataagtaatgcaactatgaatctcatggagagttcgatcctggctcaggatgaacgctggcggcatgcttaacacatgcaagtcggacgggaaacacgggaaacggtgtttccagtggcggacgggtgagtaacgcgtaagaacctgcccttgggaggggaacaacagctggaaacggctgctaataccccgtaggctgaggagcaaaaggaggaatccgcccgaggaggggctcgcgtctgattagctagttggtgaggcaatagcttaccaaggcgatgatcagtagctggtccgagaggatgatcagccacactgggactgagacacggcccagactcctacgggaggcagcagtggggaattttccgcaatgggcgaaagcctgacggagcaatgccgcgtggaggtagaaggcccacgggtcgtgaacttcttttcccggagaagaagcaatgacggtatctggggaataagcatcggctaactctgtgccagcagccgcggtaatacagaggatgcaagcgttatccggaatgattgggcgtaaagcgtctgtaggtggctttttaagtccgccgtcaaatcccagggctcaaccctggacaggcggtggaaactaccaagctggagtacggtaggggcagagggaatttccggtggagcggtgaaatgcgtagagatcggaaagaacaccaacggcgaaagcactctgctgggccgacactgacactgagagacgaaagctaggggagcgaatgggattagataccccagtagtcctagccgtaaacgatggatactaggcgctgtgcgtatcgacccgtgcagtgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccttcgggaacgcggacacaggtggtgcatggctgtcgtcagctcgtgccgtaaggtgttgggttaagtcccgcaacgagcgcaaccctcgtgtttagttgccatcgttgagtttggaaccctgaacagactgccggtgataagccggaggaaggtgaggatgacgtcaagtcatcatgccccttatgccctgggcgacacacgtgctacaatggccgggacaaagggtcgcgatcccgcgagggtgagctaaccccaaaaacccgtcctcagttcggattgcaggctgcaactcgcctgcatgaagccggaatcgctagtaatcgccggtcagccatacggcggt

SEQ ID NO: 40 - DNA, 1165 bp, Solanum tuberosum
ttcttgatcaatccctttgcccctcattcttcaagaataaggaagatccttttcaagtttgaatttgttcatttggaatctgggttcttctacttcatatttatttaatatgaatattttccctctcttttttttatatcattccttaagtcccataggtttgatcctgtagaatttgacccattttctcattgaacgaaaggtacgaaataaatcagattgataaaagtaccatgtgaaatcttcggtttttccccttcctcgatccctatcccataggtacagtgtttgaatcaatagagaaccttttcttctgtatgaatcgatattattccattccaaatccttcccgatacctcccaaggaaaatctctaatttggatcccaaattgacgggttagtgtgagcttatccatgcggttatgcactctttgaataggaatccgttttctgaaagatcctggctttcgtactttggtgggtctccgagatcctttcgatgacctatgttgaagggatatctatctaatccgatcgattgcgtaaagcccgcggtagcaacggaaccggggaaagtatacagaaaagacagttcttttctattatattagtattttctattatattagatatattagactattatattagattagtattagttagtgatcccgacttagtgagtcctttcttccgtgatgaactgttggcaccagtcctacattttgtctctgtggaccgaggagaaaaggggctcggcgggaagaggagtgtaccatgagagaagcaaggaggtcaacctctttcaaatatacaacatggattctggcaatgtagttggactctcatgtcgatccgaatgaatcatcctttccacggaggtaaatctttgcctgctaggcaagagggtagcaagttccaaattctgtctcggtaggacatgtatttctattactatgaaattcataaatgaagtagttaatggtagggttcccattatcctttttgtagtgacgaatcttgtatgtgttcctaagaaaaggaatttgtccatttttcggggtctcaaaggggcgtggaaacgcataagaactcttgaatggaaaagagatgtaactccagttccttcggaatcggtagtcaatcctatttccgataggggcag

SEQ ID NO: 41 - DNA, 1780 bp, Lycopersicon esculentum; identical to SEQ ID NO: 39

SEQ ID NO: 42 - DNA, 1185 bp, Lycopersicon esculentum
ttcttgatcaatccctttgcccctcattcttcaagaataaggaagatccttttcaagtttgaatttgttcatttggaatctgggttcttctacttcatatttatttaatatgaatattttccctctcttttttttatatcattccttaagtcccataggtttgatcctgtagaatttgacccattttctcattgaacgaaaggtacgaaataaatcagattgataaaagtaccatgtgaaatcttcggtttttccccttcctcgatccctatcccataggtacagtgtttgaatcaatagagaaccttttcttctgtatgaatcgatattattccattccaaatccttcccgatacctcccaaggaaaatctctaatttggatcccaaattgacgggttagtgtgagcttatccatgcggttatgcactctttgaataggaatccgttttctgaaagatcctggctttcgtactttggtgggtctccgagatcctttcgatgacctatgttgaagggatatctatctcatccgatcgattgcgtaaagcccgcggtagcaacggaaccggggaaagtatacagaaaagacagttcttttctattatattagtattttctattatattagatatattagactattatattagattagtattagttagtgatcccgacttagtgagtcctttcttcacttagtgagtcctttcttccgtgatgaactgttggcaccagtcctacattttgtctctgtggaccgaggagaaaaggggctcggcgggaagaggagtgtaccatgagagaagcaaggaggtcaacctctttcaaatatacaacatggattctggcaatgtagttggactctcatgtcgatccgaacgaatcatcctttccacggaggtaaatctttgcctgctaggcaagaggatagcaagttccaaattctgtctcggtaggacatgtatttctattactatgaaattcataaatgaagtagttaatggtagggttaccattatcctttttgtagtgacgaatcttgtatgtgttcctaagaaaaggaatttgtccatttttcggggtctcaaaggggcgtggaaacgcataagaactcttgaatggaaaagagatgtaactccagttccttcggaatcggtagtcaatcctatttccgataggggcag

SEQ ID NO: 43 - DNA, 1475 bp, Lactuca sativa
gggttgatccgtatcatcttgacttggttctgcttcctctatttttttaataataccgggtcgggttcttctcctacccgtatcgaatagaacacgctgagccaaatcttcttcatgtaaaacctgcttgatttagatcgggaaaatcgtgtggttttatgaaaccatgtgctatggctcgaatccgtagtcaatcctatttccgatagggacagttgacaactgaatcctattttcccattattttcatatccgtaatagtgcgaaaaaaaagaaggcccggccccaagttgttcaagaatagtgtcgttgagtttctcgaccctttgccttaggattaatcagttctatttctcgatgggggcagggaagggatataactcagcggtagagtgtcaccttgacgtggtggaagtcatcagttcgagcctgattatccctaaacccaatgtgagttttgatattttgatttgctaccccgccgtgattgaatgagaatggataagaggctcgtgggattgacgtgagggggcagggatggctatatttctgggagcgaactccgggcgaatatgaagcgcatggatacaagttaggccttggaatgaaagacaattccgaatccgctttgtctacgaacaaggaagctataagtaatgcaactatgaatctcatggagagttcgatcctggctcaggatgaacgctggcggcatgcttaacacatgcaagtcggacgggaagtggtgtttccagtggcggacgggtgagtaacgcgtaagaacctgcccttgggaggggaacaacagctggaaacggctgctaataccccgtaggctgaggagcaaaaggaggaatccgcccgaggaggggctcgcgtctgattagctagttggtgaggtaatagcttaccaaggcgatgatcagtagctggtccgagaggatgatcagccacactgggactgagacacggcccagactcctacgggaggcagcagtggggaattttccgcaatgggcgaaagcctgacggagcaatgccgcgtggaggtagaaggcccacgggtcatgaacttcttttcccggagaagaagcaatgacggtatctggggaataagcatcggctaactctgtgccagcagccgcggtaatacagaggatgcaagcgttatccggaatgattgggcgtaaagcgtctgtaggtggctttttaagtccgccgtcaaatcccagggctcaactctggacaggcggtggaaactaccaagctggagtacggtaggggcagagggaatttccggtggagcggtgaaatgcgtagagatcggaaagaacaccaacggcgaaagcactctgctgggccgacactgacactgagagacgaaagctaggggagcgaatgggattagataccccagtagtcctagccgtaaacgatggatactaggcgctgtgcgtatcgacccgtgcagtgct

SEQ ID NO: 44 - DNA, 1428 bp, Lactuca sativa
tttttcgatcaatccctttgcccctcattcttcgagaatcagaaagatttttttcaagtttgaatttgttcatttggaatctgggctcttctttatttattattttatttattatttttccctctcttttctttttttattcccttccgtcattccttaagtcccataggtttgatcctgtagaatctgacccactttctcattgagcgaagggtacgaaataaatcagattgatttttcgatcaaaagtactatatcaaaagtactatgtgaaatcttcggttttttcctctttcgctaccccttacagcgtttgaatcaatagagaaccttttcttctctatctgtatgaatcgatattattacattccaattccttcccgacacctcccaaggaaaatcctgaattggatcccaaattgacgggttagtgtgagcttatccatgcggttatgcactcttcgaataggaatccattttctgaaagatcctggctttcgtgctttggtgggtctccgagatcctttcaatgacttatgttgtgttgaaaggatatctatatgatccgatcgattgcgtaaggcacgcggtagcaacggaaccggggaaagtatacagaaaagacagttcttttctattctattagtattagattaggattagttagtattagattagtattagttagtgatcccggctcagtgagtcctttcttccgggatgaactgttggtaccagtcctacattttgtctctgtggaccgaggagaaatccgcctctttcaaatatacaacatggattctggcaattcaatggagttggactctcatgtcgatccgaatgaatcatcctttccacggaggtaaatctttgcctgctaggcaagaggatagcaagttacaaattctgtctcggtaggatatacatgtattttatttctattactatgaaattcataaatgaagtagttaataaatgaaataaaatagttaagggtggggttaccattatcctttttgtagtgatgaatcttgtatgtgttcctaagaaaaggaatttgtccatttttcggggtctcaaaggggcgtggaaacacataagaactcttgaatggaaatggaaaagagatgtaactccagttccttcggaaatggtaagatctttggcgcaagaagaaggggttgatccgtatcatcttgacttggttctgatttctctatttttttaagaataccgatttaagaataccgagtcggggtcttctcctacctgtatcgaatagaacatgctgagccaaatcttcttcatgtaaaacctgctttatttaatgtaaaacctgctttatttagatcgggaaaatcatatgcttttttgaaaccatgtgctatggctcgaatccgtagtcaatcctattttcgataggagcag

SEQ ID NO: 45 - DNA, 1770 bp, Populus alba
ttgacaattgaatccaattttcccattattttcgtatccgtaatagtgcgaaaagagggcccgactccaagttgttcaagaatagtggcgttgagtttctcgaccctttgacttaggattagtcagttctatttctcgatgggggcaagggaagggatataactcagcggtagagtgtcaccttgacgtggtggaagtcatcagttcgagcctgattatccctaaacccaatgtgagtttttctattttgacttgctcaccctccgccgtgatcgaatgagaatggataagaggctcgtgggattgacgtgagggggtagggatggctatatttcttggagcgaactctaagcgaatatgaagcgcatggatacaagccttgaaatgaaagacaattccgaatcagctttgtctacgaacaaggaagctataagtaatgcaactaagaatctcatggagagttcgatcctggctcaggatgaacgctggcggcatgcttaacacatgcaagtcggacgggaagtggtgtttccagtggcggacgggtgagtaacgcgtaagaacctgcccttgggaggggaacaacagctggaaacggctgctaataccccgtaggctgaggagcaaaaggaggaatccgcccgaggaggggctcgcgtctgattagctagttggtgaggcaatagcttaccaaggcgatgatcagtagctggtccgagaggatgatcagccacactgggactgagacacggcccagactcctacgggaggcagcagtggggaattttccgcaatgggcgaaagcttgacggagcaatgccgcgtggaggtagaaggcctacgggtcgtgaacttcttttcccggagaagaagcaatgacggtatctggggaataagcatcggctaactctgtgccagcagccgcggtaatacagaggatgcaagcgttatccggaatgattgggcgtaaagcgtctgtaggtggctttttaagtccgccgtcaaatcccagggctcaaccctggacaggcggtggaaactgccaagctggagtacggtaggggcagagggaatttccggtggagcggtgaaatgcgtagagatcggaaagaacaccaacggtgaaagcactctgctgggccgacactgacactgagagacgaaagctaggggagcgaatgggattagataccccagtagtcctagccgtaaacgatggatactgggcgctgtgcgtatcgacccgtgcagtgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccttcgggaacgcggacacaggtggtgcatggctgtcgtcagctcgtgccgtaaggtgttgggttaagtcccgcaacgagcgcaaccctcgtgtttagttgccaccgttgagtttggaaccctgaacagactgccggtgataagccggaggaaggtgaggatgacgtcaagtcatcatgccccttatgccctgggcgacacacgtgctacaatggccgggacaaagggtcgcgatcccgcgagggtgagctaactccaaaaacccgtcctcagttcggattgcaggctgcaactcgcctgcatgaagccggaatcgctagtaatcgccggtcagccatacggcggt

SEQ ID NO: 46 - DNA, 1428 bp, Populus alba; identical to SEQ ID NO: 44

SEQ ID NO: 47 - DNA, 1781 bp, Brassica
accgccgtatggctgaccggcgattactagcgattccggcttcatgcaggcgagttgcagcctgcaatccgaactgaggacgggtttttggagttagctcaccctcgcgggatcgcgaccctttgtcccggccattgtagcacgtgtgtcgcccagggcataaggggcatgatgacttgacgtcatcctcaccttcctccggcttatcaccggcagtctgttcagggttccaaactcaacggtggcaactaaacacgagggttgcgctcgttgcgggacttaacccaacaccttacggcacgagctgacgacagccatgcaccacctgtgtccgcgttcccgaaggcacccctctctttcaagaggattcgcggcatgtcaagccctggtaaggttcttcgctttgcatcgaattaaaccacatgctccaccgcttgtgcgggcccccgtcaattcctttgagtttcattcttgcgaacgtactccccaggcgggatacttaacgcgttagctacagcactgcacgggtcgatacgcacagcgcctagtatccatcgtttacggctaggactactggggtatctaatcccattcgctcccctagctttcgtctctcagtgtcagtgtcggcccagcagagtgctttcgccgttggtgttctttccgatctctacgcatttcaccgctccaccggaaattccctctgcccctaccgtactcaagcttggtagtttccaccgcctgtccagggttgagccctgggatttgacggcggacttaaaaagccacctacagacgctttacgcccaatcattccggataacgcttgcatcctctgtattaccgcggctgctggcacagagttagccgatgcttattccccagataccgtcattgcttcttctctgggaaaagaagttcaggacccgtaggccttctacctccacgcggcattgctccgtcaggctttcgcccattgcggaaaattccccactgctgcctcccgtaggagtctgggccgtgtctcagtcccagtgtggctgatcatcctctcggaccagctactgatcatcgccttggtaagctattgcctcaccaactagctaatcagacgcgagcccctcctcgggcggattcctccttttgctcctcagcctacggggtattagcagccgtttccagctgttgttcccctcccaagggcaggttcttacgcgttactcacccgtccgccactggaaacaccacttcccgtccgacttgcatgtgttaagcatgccgccagcgttcatcctgagccaggatcgaactctccatgagattcatagttgcattacttatagcttccttcttcgtagacaaagctgattcggaattgtctttcattccaagtcataacttgtatccatgcgcttcatattcgcatggagttcgctcccagaaatatagctacccctaccccctcacgtcaatcccacgagcctcttatccattcttattcgatcacagcgagggagcaagtcaaaatagaaaaactcacattcattgggtttagggataatcaggctcgaactgatgacttccaccacgtcaaggtgacactctaccgctgagttatatcccttcccccatcaagaaatagaactgactaatcctaagtcaaagggtcgagaaactcaaggccactattcttgaacaacttggattggagccgggctttcctttcgcactattacgggtatgaaatgaaaataatggaaaaagttggattcaattgtcgg

SEQ ID NO: 48 - DNA, 1235 bp, Brassica
cttgcgccaaagatcttcctatttccaaaggaactggagttacatctcttttccatttccattcaagagttcttatgtgtttccacgcccctttaagaccccgaaaaattaacaaattcccttttcttaggaacacgtgcgagataaaaaaaaaaagagagaatggtaaccccacgattaactatttcatttatgaatttcatagtaatagaaatacatgtcctaccgaaacagaatttgtaacttgctatcctataatcttgcctagcaggcaaagatttcactccgcgaaaaagatgattcattcggatcaacatgaaagcccaactacattgcattgccagaattcatgttatctattggaaagaggttgacctccttgcttctatggtacaatcctcttcccgctgagcctcctttcttccgtgattaactgttggcaccagtcctacattttgtctctgtggaccgagaagaaaggactcactgcgccaagatcactaactaacactaatctaatagaatagaaaatcctaatataatagaaaagaactgtcttttctgtatacttatgtatactttccccggttccgttgctactgcgggctttacgcaatcgatcggatcatctagatatcccttcaacacaacataggtcgtcgaaaggatctcggagacccgccaaagcacgaaagccaggatctttcagaaaataaattcctattcgaagagtgcataaccgcatggataagctcacactaacccgtcaatttgggatccaattcgggattttccttgagggatattggtaaggaattggaatgtaataatatcgattcataatggattcatatcgatacagaagaaaaggttctctatcgattcaacaagtgctgtacttatgggaaagcgatagagaaagagaaaaaaaaaacgaagatttcacatagtgatttttttttgatcaaaaaaaaatatgattgaatttatttcgtacccttcgctcaatgagaacatgggtcagattctataggatcaaacctatgggacttaagaatgatggaagggaataaaatcaaaaaagaaatcaaataaagaaaagagagggaaaataaagaaataataagtaaataaaaatgaagtagaagaacccagattacaaatgaacaaattcaaacttgaaaaagtctctttctgattctcgaagaatgaggggcaaagagattgatcgagaa

SEQ ID NO: 49 - DNA, 1781 bp, Lemna gibba
ttgacaattgaatcccatttttcccattatttcactatacgtaatagtgcgaaaagaaggcccggctacaagttaagttgtttaggaatagtggaatagtggcgttgagtttctctaccctttgacttaggattagtcagttctatttctcgataggggccgggaagggatataactcagcggtagagtgtcaccttgacgtggtggaagtcatcagttcgagcctgattatccctaaacccaatgtgagtttttctattttgacttgctcccccggagtgatcgaacgagaatgaataagaggctcgtgggattgacgtgatagggtagggatggctatattgctgggagcgaactccaggctaatatgaagcgcatggatacaagttatgccttggaatgaaagacaattccgaatcagctttgtctacgaacaaggaagctataagtaatgcaactatgaatctcatggagagttcgatcctggctcaggatgaacgctggcggcatgcttaacacatgcaagtcggacgggaagtggtgtttccagtggcggacgggtgagtaacgcgtaagaacctgcccttgggaggggaacaacaactggaaacggttgctaataccccgtaggctgaggagcaaaaggaggaatccgcccaaggaggggctcgcgtctgattagctagttggtgaggtaatagcttaccaaggcgatgatcagtagctggtccgagaggatgatcagccacactgggactgagacacggcccagactcctacgggaggcagcagtggggaattttccgcaatgggcgaaagcctgacggagcaatgccgcgtggaggtagaaggcccacgggtcgtgaacttcttttctcggagaagaagcaatgacggtatctgaggaataagcatcggctaactctgtgccagcagccgcggtaagacagaggatgcaagcgttatccggaatgattgggcgtaaagcgtctgtaggtggcttttcaagtccgtcgtcaaatcccagggctcaaccctggacaggcggtggaaactaccaagctggagtacggtaggggcagagggaatttccggtggagcggtgaaatgcgtagagatcggaaagaacaccaatggcgaaagcactctgctgggccgacactgacactgagagacgaaagctaggggagcgaatgggattagataccccagtagtcctagccgtaaacgatggatactaggcgctgtgcgtatcgacccgtgcagtgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccttcgggaacgcggacacaggtggtgcatggctgtcgtcagctcgtgccgtaaggtgttgggttaagtcccgcaacgagcgcaaccctcgtgtttagttgccaccattgtggaaccctgaacagactgccggtgataagccggaggaaggtgaggatgacgtcaagtcatcatgccccttatgccctgggcgacacacgtgctacaatggccgggacaaagggtcgcgatcccgcgagggtgagctaaccccaaaaacccgtcctcagttcggattgcaggctgcaactcgcctgcatgaagccggaatcgctagtaatcgccggtcagccatacggcggt

SEQ ID NO: 50 - DNA, 1372 bp, Lemna
ttcgagaatctcctcccttcttccactccgtcccgaagagtaactaacaccaattcagtcacgttttcatgttccaattgaacactttccatttttgattatgatcgaaggagaagattatttaccaaacatatgcggatcaaatcacgatattataattaagaacaagagatctttctcgatcaatccctttgtccctcagtcttcgagaataagaaagatcctttgtgaaatcttcggtttttttcctcttcctctatcccataggtacatgaatcaatagagaaccttttcttctgttctgtatgaatcgatattattacattccaattctttcccgataccttccaaggaaaatctcaaattggatccaaaaaaaatgaacgggttggtgtgagcttatccatgcggttatgcactcttcaaataggaatccattttctgaaagattctggctttcgtgctttggtgagtcgtccgagatcttttcgatgacctatgttgtgttgaagggatatctatatgatccgatccattgcgtaaggcccgcggtacggtagcaacggaaccggggaaaatatacagaaaagagagttcttttctattcttttctattatattagtattagttagttaccccggctcggtgagtcttttcttccgtgatgaactgttggtaccagtcctatattttttctctgtggaccgaagataaagggggctccgcagagggttgtaccatgaagagaagcaaggaggtcaacctgtttaaaatatacaacatggattctagcaatgcaatgtagttggaccctcatgtcgatccgaattaatcagtctttccacggaagtctatttttgcctgctaggcaagaggataacaagttcaaaattctgtctcggtaagacatgtatttctattactaataaattcataaatgaagtagttaatttaatggtggggttaccattatccttttgtagtgacgaatcttgcatgtgttcctaagaaaaggaattagtccatttcatttttgtttgaggtttcaaaaaaaggctgtggaaatacatagaactcttgaatggaaattgaaaagagatgtagctacagttccttcggaaatggtaagatctttggcgcaagaagaacgggttgatccgtatcatcttgacttggttctgcttcctctccttttttttttaacaataccgagtcgggttcttctcctaccagtatcgaatagaacatgctgaacaaaatatcttcttcatgtaaaacctgctctatttagatcggaaaaatcgtacggattttatgaaaccatgtgctatggctcgaatccatagtcaatcctatttccgataggagcat

SEQ ID NO: 51 - DNA, 1765 bp, Glycine max
accgccgtatggctgaccggcgattactagcgattccggcttcatgcaggcgagttgcagcctacaatccgaactgaggacgggtttttggagttagctcaccctcgcgggatcgcgatcctttgtcccgtccattgtagcacgtgtgtcgcccagggcataaggggcatgatgacttgacgtcatcctcaccttcctccggcttatcaccggcagtctgctcagggttccaaactaaatgttggcaactaaacacgagggttgcgctcgttgcgggacttaacccaacaccttacggcacgagctgacgacagccatgcaccacctgtgtccgcgttcccgaaggcacccctctctttcaagaggattcgcggcatgtcaagccctggtaaggttcttcgctttgcatcgaattaaaccacatgctccaccgcttgtgcgggcccccgtcaattcctttgagtttcattcttgcgaacgtactccccaggcgggatacttaacgcgttagctacagcattgcacgggtcgatacgcacagcgcctagtatccatcgtttacggctaggactactggggtatctaatcccattcgctcccctagctttcgtctctcagtgtcagtgtcggcccagcagagtgctttcgccgttggtgttctttccgatctctacgcatttcaccgctccaccggaaattccctctgcccctaccgtactccagcttggtagtttccaccgcctgtccagggttgagccctgggatttgacggcgaacttaaaaagccacctacagacgctttacgcccaatcattccggataacgcttgcatcctctgtcttaccgcggctgctggcacagagttagccgatgcttattccccggataccgtcattgcttcttctccgggaaaagaagttcatgacccgtaggccttctaccttcacgcggcattgctccgtcaggctttcgcccattgcggaaaattccccactgctgcctcccgtaggagtctgggccgtgtctcagtcccagtgtggctgatcatcctctcggaccagctactgatcatcgccttggtaagctattgcctcaccaactagctaatcagacgcgagcccctcctcgggcggattcctccttttgctcctcagcctacggggtattagcagccgtttccagctgttgttcccctcccaagggtaggttcttacgcgttactcacccgtccgccactggaaacaccacttcccgtccgacttgcatgtgtaaggcatgccgccagcgttcatcctgagccaggatcgaactctccatgagattcctagttgcattacttatagcttccttgttcgtagacaaagctgattcggaattctcttccattccaaggcataacttgtatccaggcgcttcatattcgactggagttcgctcccagaaatatagcccccccacccctcgtgtaatcccacgagcctcttatccattctcaattattattcgatcacggcatacaaatagaaaaacttacattgggtttagggataatcaggctcgaactgataacttccaccacgtcaaggtgacactctaccgctgagttatatcccttgcctccatcgagaaatagaactgactaatcctaaggcaaagggtcaagaaactcaacgccactattcttaaacaacttcttggagttaggctttcttttcacactattacggatacgaaaattatgggaaaaattggattccattgtcaa

SEQ ID NO: 52 - DNA, 1322 bp, Glycine max
ctgctcctatcggaaataggattgaccacggattcgagcaatagcacatgatttcataaaaccatatgattttcccgatctaaataaagcaggttttccatgaagaagatttggctaagcatgttctattcgatacgggtaggagaagaacccgactcgattttattaaaaaatagagaaatcagaaccaagtcaagatgatacggatcaacccctcctcttgcgttcttgcgccaaagatcttaccatttccgaagtaactggagttacatctatttttccatttcaattcaagagttcttatgtgtttccacgccccttcgagaccccgaaaaaatggacaaattgcttttcttaggaacacatattcgttaccacaaaaaggataatggtaaccccaccattaactacttcaatttaatagtaataaaaatacatgccctaccaagacagaatttgtaatttgctatactcttgcctagcaggcaaagatttacctccgtggaaaggatgattcattcagatcgacatgagagtccaaccacattgcattgccagaacccatgttgtatatttgaaaaaggttgacctccttgcttctctcatgttacaatcctcttgccgctgagctccctttctcctcggtccacagagacaaaatgtaagacaggtgccaacagttcatcacgaaagaaaggactagctgagccgagatcactaactaaaacgaatttaatactaatcgaaaatactaatagaatagaaatagaactgtcttttcatatagatatcccttcaacacaacataggtcatcgaaaggatctcgaagacccaccaacgcacgaaagccgggatctttcagaaaatggattcctattcgaagggtgcataaccgcatggataagctcacactaacccgtcaatttgggatccaattcggttccttaggaggtatcgggaaggaattttggaatgtaataatatcgattcatacagatacagaagaaagggttctctattgattcaaacgctctacctacgagatagggatagagaaagaggaaaaaaccgaggatttcacatagtacttttggtcgaaaaatcgatctgatttttttcgtacttttcgctcaatgatgaaatgggtcagattctacaggaccaaaccttgtgggagttaaggaatgatggaagaaaagaaataaaaaaaagtaaataaaaatgcagtagaagagtcctgattccaaacgaaaaaattcaaacttgattgaaaaggatctttctgattattgaagaatgggacaaaaggattgatcgcgaa

SEQ ID NO: 53 - DNA, 1761 bp, Oryza sativa
ttgacaattgaatccaatttttcccattatttgactatccataatagtgtggaaagaaagcccggaggaagggtggccttgagtttctcgcccctttgccttaggattcggtaattctctttctcgaggggacggggaagggatataactcagcggtagagtgtcaccttgacgtggtggaagtcatcagttcgagcctgattatccctaaacccaatgtgagttttttctattttgacttactccccccgccacgatcgaacgggaatggataagaggcttgtgggattgacgtgatagggtagggttggctatactgctggtggcgaactccaggctaataatctgaagcgcatggatacaagttatccttggaaggaaagacaattccgaatccgctttgtctacgaataaggaagctataagtaatgcaactatgaatctcatggagagttcgatcctggctcaggatgaacgctggcggcatgcttaacacatgcaagtcgaacgggaagtggtgtttccagtggcgaacgggtgagtaacgcgtaagaacctgcccttgggaggggaacaacaactggaaacggttgctaataccccgtaggctgaggagcaaaaggagaaatccgcccaaggaggggctcgcgtctgattagctagttggtgaggcaatagcttaccaaggcgatgatcagtagctggtccgagaggatgatcagccacactgggactgagacacggcccagactcctacgggaggcagcagtggggaattttccgcaatgggcgaaagcctgacggagcaatgccgcgtggaggtggaaggcccacgggtcgtcaacttcttttctcggagaagaaacaatgacggtatctgaggaataagcatcggctaactctgtgccagcagccgcggtaagacagaggatgcaagcgttatccggaatgattgggcgtaaagcgtctgtaggtggcttttcaagtccgccgtcaaatcccagggctcaaccctggacaggcggtggaaactaccaagctggagtacggtaggggcagagggaatttccggtggagcggtgaaatgcattgagatcggaaagaacaccaacggcgaaagcactctgctgggccgacactgacactgagagacgaaagctaggggagcaaatgggattagagaccccagtagtcctagccgtaaacgatggatactaggtgctgtgcgactcgacccgtgcagtgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccctcgggaacgcggacacaggtggtgcatggctgtcgtcagctcgtgccgtaaggtgttgggttaagtctcgcaacgagcgcaaccctcgtgtttagttgccactatgagtttggaaccctgaacagaccgccggtgttaagccggaggaaggagaggatgaggccaagtcatcatgccccttatgccctgggcgacacacgtgctacaatgggcgggacaaagggtcgcgatctcgcgagggtgagctaactccaaaaacccgtcctcagttcggattgcaggctgcaactcgcctgcatgaagcaggaatcgctagtaatcgccggtcagccatacggcggt

SEQ ID NO: 54 - DNA, 1304 bp, Oryza sativa
tctcggtatcaatccccttgcccctcattctttgagaatcagaaggatccttttcgagtttccatttcttcatttggaatctgggctcttctatcttcgacttattttttttttgctttattctttatttatttcatttcgatttttccctcttcctctatccctatcctctaggtacagcgtttgcatcaatagagaaccttttcctctgtatgaatctatattattccattccaatttcttcccgaaacttcccaagaaaaatcccgaattggatccaaaattgacgggttaatgtgagcttatccatgcggttaggcactcttcaaataggaatccattttctaactggctttcgtgctttggtgagtcgtccgagatcctttcgatgacctatgttgtgttgaagggatatctatatgatccgatcgattgcataagacccgcggtagcaatagaacggggaaagtatacagaaaagacagttcttttcaatttcgattatctatatattagttgtttctatttctagatatctatttctatatattagtattagttagtagtactattctattagttagcgatcccggctctgtgagttctttcttccgtgatgaactgtcggcaccagtcctccattttttctctgtggaccgaggagaaagggggctcagcaggaagaggattgtaccatgagagaagcacagaggtcaaccctcttcaaatatggaacatggattctgcaatgcaacgtagttgggtcctcatatcgatccgaatgaatcattctttctacagaggtcaatctttgcctattaggcaagaggatagcaagttcgaaattctgtctcggtaggacatggatttctattactatgaaattcataaatgaaatgaagtagttaatgggagggctaccattatcctttttcttgtatgtgttcctaagagaaggaatttgtccatttcatgtttcgaggtctcaaaaaagggcgtggaaacagataagaaactcttgaatggaaattgaaaagaaatgtagccccagttccttcggaaatggtaagatctttggcgcaagaagaaggggcgacccatatcatcttgacttggttctgcttcccctctttttttaagaataccgagtcgggttcttctcctaccagtatcgaatagaacatgctgaacaagatcttcttcatggaaacctgctcgatttagatcgggaaaatcgtacagattttatgaaaccatgtgctatggctcgaatccatagtcaatcctattttcgataggaccgg

(19) United States
(12) Patent Application Publication
(10) Pub. No.: US 2009/0172832 A1
(43) Pub. Date: Jul. 2, 2009

(54) EXPRESSION OF RUBISCO ENZYME FROM A NON-RUBISCO LOCUS

(76) Inventors: TIMOTHY CASPAR, Yorklyn, DE (US); Theodore Mitchell Klein, Wilmington, DE (US); Min Qi, Hockessin, DE (US); Jianjun Yang, Hockessin, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 12/204,005

(22) Filed: Sep. 4, 2008

Related U.S. Application Data
(60) Provisional application No. 61/017,422, filed on Dec. 28, 2007.

(51) Int. Cl. C12N 15/82; C12N 5/10

(52) U.S. Cl. 800/278; 435/419

(57) ABSTRACT

The invention relates to a method for transformation of plant chloroplasts with genetic constructs by insertion of a RUBISCO gene in a non-RUBISCO site of the chloroplast genome to generate transformed plants that produce large amounts of a functional RUBISCO enzyme.

This application claims the benefit of U.S. Provisional Application 61/017422 filed, Dec. 28, 2007

### FIELD OF THE INVENTION

The invention relates to the field of molecular biology and plant genetics. More specifically the invention relates to a method for transformation of plant chloroplasts with genetic constructs that express foreign proteins at very high levels. More specifically, the invention relates to a method for transformation of plant chloroplasts that produce very high levels of RUBISCO enzyme to improve photosynthesis for better crop performance.

### BACKGROUND OF THE INVENTION

The initial step of the photosynthetic fixation of carbon dioxide (CO₂), the carboxylation of ribulose-1,5-bisphosphate (RuBP), is catalyzed by the most abundant enzyme on the earth, ribulose-1,5-bisphosphate carboxylase/oxygenase (RUBISCO, E. C. 4.1.1.39). The reaction products are two molecules of 3-phospho-glycerate (PGA), which are partly utilized in the Calvin cycle to regenerate the carbon dioxide acceptor, RuBP, and partly converted to carbohydrate which supports plant growth. This pathway is responsible for the annual net fixation of 10¹¹ tons of CO₂ into the biosphere, a process upon which all agriculture ultimately depends. In addition to carboxylation of RuBP, RUBISCO also catalyzes its oxygenation, producing one molecule of PGA and one molecule of phosphoglycolate from each molecule of RuBP. The PGA is recycled through the Calvin cycle but the phosphoglycolate is metabolized by the photorespiratory pathway. This pathway utilizes energy in the form of ATP and reducing equivalents to recycle three quarters of the carbon in the phosphoglycolate back to PGA. However, for each molecule of RuBP which is oxygenated, one half molecule of CO₂ is released during photorespiration. The oxygenation reaction of RuBP performed by RUBISCO has no widely accepted value to the plant. Similarly, with the exception of recycling phosphoglycolate back into PGA, the photorespiratory pathway also has no known value to the plant.

The RUBISCO enzyme from plants is a sub-optimal enzyme because of its low catalytic activity and poor ability to discriminate between CO₂ and O₂ (Andrews, T. J., Whitney, S. M., Arch Biochem Biophys, 414, 159-169, 2003). Models which relate RUBISCO parameters to photosynthesis, growth, and yield (von Caemmerer, S., Biochemical Models of Leaf Photosynthesis 2000, CSIRO Publishing; Zhu, X.-G., et al., Plant Cell and Environment, 27, 155-165, 2004 ; Alagarswamy, G., et al., Agron. J., 98, 34-42, 2006; Whitney, S. M. and Andrews, T. J., Plant Physiol., 133, 287-294, 2003) predict that increasing RUBISCO's catalytic efficiency will result in a substantial increase in plants' productivity. In particular, if the oxygenase activity were eliminated and the rate of carboxylation increased about ten-fold, plant productivity would be predicted to increase by 50%.

A RUBISCO with better kinetic properties than that in a target plant could be identified from other plants or from non-plant sources. Alternatively, an improved RUBISCO enzyme could be created by rational protein design and/or in vitro evolution (e.g. U.S. patent application Ser. No. 09/437,726 and US patent No. 2006/0117409A1). Chloroplast transformation could be used to introduce RUBISCO enzymes into a target plant to improve photosynthesis. The sunflower RUBISCO large subunit (LSU) gene (Kanevski, I., et al., Plant Physiol., 119, 133-141, 1999) and the microbial RUBISCO LSU gene from Rhodospirillum rubrum (Whitney, S. M. and Andrews, T. J., Plant Physiol., 133, 287-294, 2003 and Whitney, S. M. and Andrews, T. J., Proc. Natl. Acad. Sci. (USA), 98, 14738-14743, 2001) have been introduced into the chloroplast genome of tobacco. In this work, the entire sunflower LSU and a fusion protein containing the N-terminus of the tobacco LSU with the entire Rhodospirillum rubrum LSU were synthesized in the transplastomic tobacco. The RUBISCO large subunit (rbcL) and small subunit (rbcS) genes from the red alga Galdieria sulphuraria and the diatom Phaeodactylum tricornutum have also been introduced into the inverted repeats of the chloroplast genome of tobacco (Whitney S. M., et al., Plant J., 26, 535-547, 2001). Large amounts of Galdieria sulphuraria and Phaeodactylum tricornutum RUBISCO proteins were expressed from these transgenes, however they were not properly assembled into a functional holoenzyme.

The problem to be solved therefore is to: 1) achieve functional expression of high levels of the RUBISCO genes in a non-RUBISCO site and 2) provide a method for transformation of plant chloroplasts with a RUBISCO enzyme having improved kinetic properties. Expression of this enzyme will improve photosynthetic carbon fixation ultimately leading to better crop performance. In order to be agronomically useful, such a method must express a foreign RUBISCO at substantial levels. For example, a RUBISCO with a k^{c}_{cat} equivalent to the plant enzyme (˜3 s⁻¹) will need to be expressed at approximately the same levels as the endogenous RUBISCO, or about 50% of the soluble leaf protein. For enzymes with higher k^{c}_{cat} values, the requisite expression level will be somewhat lower.

### SUMMARY OF THE INVENTION

This invention discloses a plant cell having a genetic construct inserted in a non-RUBISCO region of the chloroplast genome where the genetic construct encodes a heterologous RUBISCO enzyme. Preferred insertion loci are in the inverted repeat regions of the chloroplast genome. Surprisingly, using a novel vector and non-RUBISCO regulatory elements, expression of the RUBISCO enzyme from these loci resulted in production of high levels of functional and soluble enzyme. Neither of these attributes has previously been used for successful expression of functional, soluble RUBISCO holoenzymes in plants.

Accordingly, the invention provides a plant cell comprising a chloroplast genome having inserted therein a heterologous genetic construct encoding a RUBISCO enzyme, wherein the genetic construct is inserted at a non-RUBISCO locus in the genome and wherein the RUBISCO enzyme is selected from the group consisting of: a RUBISCO large subunit and a RUBISCO small subunit. Preferred chloroplast loci for the expression of the genetic construct are within the inverted repeat region of the chloroplast genome.

In another embodiment the invention provides a method for the expression of a RUBISCO enzyme in a plant comprising:

a) Providing a plant comprising a chloroplast genome;

b) Providing a vector consisting essentially of the general structure:

HA1-hetero Pro1::M::Ter1 hetero Pro2::RBC::Ter2-HA2

Wherein:
i) hetero Pro1 is a first promoter derived from a non-RUBISCO plant gene;
ii) M is a genetic construct encoding a selectable marker;
iii) Ter1 is a first terminator;
iv) hetero Pro2 is a second promoter derived from a non-RUBISCO plant gene;
v) RBC is a genetic construct encoding a plant protein selected from the group consisting of: the small subunit of a RUBISCO enzyme and the large subunit of a RUBISCO enzyme;
vi) Ter2 is a second terminator;
vii) HA1 is a first homology arm having homology to a first section of the inverted repeat region of the chloroplast genome; and
viii) HA2 is a second homology arm having homology to a second section of the inverted repeat region of the chloroplast genome; and

c) transforming the plant of (a) with the vector of (b) wherein the vector inserts in the inverted repeat region of the chloroplast genome and the RBC is a genetic construct that is expressed producing a RUBISCO enzyme in soluble form.

### BRIEF DESCRIPTION OF THE FIGURES AND SEQUENCE LISTING

The invention can be more fully understood from the following detailed description and the accompanying drawings and Sequence Listing which form a part of this application.

FIG. 1—Shows the map of pTCP10

FIG. 2—Shows the plasmid map of pTCP101

FIG. 3—Shows the plasmid map of pTCP102

FIG. 4—Shows a PCR analysis demonstrating that plants 81021, 81311, and 81321 have been transformed. PCR assay for transgene insertion in transformants of pTCP102. A 6.3 kb product indicates transgene integration from pTCP102 into the chloroplast genome. A 3.1 kb product is derived from a chloroplast genome fragment without the transgene insertion. Plant ID numbers are shown on the top. KOrbcL-KO plant. Sizes of PCR products are marked on the right.

FIG. 5−Shows an RT-PCR analysis demonstrating expression of cpNTrbcL transcript in the 81021, 81311, and 81321 plants and its absence in the 81051 and rbcL-KO plants. A 252 bp fragment represents the cpNTrbcL transcript. Plant ID numbers are shown on the top. KO: rbcL-KO plant. Size of PCR products is marked on the right.

FIG. 6—Is a Coomassie Blue stained sodium dodecyl sulfate (SDS)-PAGE gel showing that plants 81021, 81311, and 81321 had accumulated substantial amounts of RUBISCO LSU, up to levels comparable to the wild type tobacco, while plants 81051 and rbcL-KO did not contain any detectable levels of RUBISCO LSU.

FIG. 7—Is the result of SDS-PAGE western blot analysis showing that the accumulated LSU in plants 81021, 81311 and 81321 was a product of the cpNTrbcL transgene since it had a 6-His tag, while plants 81051 and rbcL-KO did not show His-tagged LSU accumulation.

FIG. 8—Shows a native-PAGE gel (stained with Coomassie Blue). Accumulation of significant amounts of a protein complex with the same size as the 550 kD L₈S₈ RUBISCO holoenzyme of wild type tobacco in plants 81021, 81311, and 81321 can be seen. However, the plants 81051 and the rbcL-KO did not show accumulation of this complex.

FIG. 9—Depicts western blot analysis using Anti-His (C-term)-HRP Antibody. The 550 kD complex that accumulated in plants 81021, 81311, and 81321 contained the LSU-6His tag.

FIG. 10—Shows a SDS-PAGE gel analysis of protein profiles during purification. In addition to LSU-6His the endogenous SSU was purified.

FIG. 11—Shows western blot analyses of purified LSU-6His protein and its complex. Using the Anti His-tag antibody confirmed that the LSU-6His was purified (upper right panel) and it was located in a 550 kD RUBISCO complex (lower right panel). Using Anti SSU antibody confirmed that the endogenous SSU was co-purified (upper left panel) and formed a complex (lower left panel) with LSU-6His.

FIG. 12−Shows the plasmid map of pTCP15

FIG. 13—Shows the plasmid map of pTCP107

FIG. 14—Shows PCR and Southern analyses of the transgene insertion into the chloroplast genome. FIG. 14A shows amplification of two products with equal intensity from the DNA preparation. The presence of a 6.3 kb fragment demonstrated that the correct transgene insertion had occurred in this plant. A shorter 3.1 kb fragment indicated that there might still be some non-transformed plastomes in the plant. FIG. 14B shows that only a larger 4.3 kb fragment was detected from the DNA sample of plant 1074, which demonstrated that all or at least the great majority of plastomes had been transformed in this plant.

FIG. 15—Protein profiles on a Coomassie Blue stained SDS-PAGE gel. Crude leaf extracts (5 mg protein) of pTCP107 transformant 1074, WT, and rbcL KO tobacco and purified R. rubrum RUBISCO (2 mg) were subjected to SDS-PAGE and then Coomassie Blue stain. Plant ID numbers are shown on the top. WT: wild type plant. KO: rbcL-KO plant. RRrbcM: purified rubrum RUBISCO. LSU: RUBISCO large subunit. SSU: RUBISCO small subunit.

FIG. 16—Shows a SDS-PAGE western blot analysis of crude leaf extracts of transformant 1074, wild type and rbcL-KO tobacco and purified R. rubrum RUBSICO confirming that the RUBISCO-like protein that accumulated in plant 1074 was the R. rubrum RUBISCO protein. Tobacco RUBISCO LSU was not detected in the same assay.

FIG. 17—Shows a Coomassie Blue stained Native-PAGE gel visualizing the L8S8 RUBISCO complex in wild-type plant and L2 RUBISCO-like complex in plant 1074.

FIG. 18—Shows a Native-PAGE gel western blot analysis. The L₂-like complex consisted of the expressed R. rubrum RUBISCO protein recognized by anti-R. rubrum RUBISCO antibody.

The sequence descriptions and Sequence Listing attached hereto comply with the rules governing nucleotide and/or amino acid sequence disclosures in patent applications as set forth in 37 C.F.R. §1.821-1.825.

The Sequence Listing contains the one letter code for nucleotide sequence characters and the one letter codes for amino acids as defined in conformity with the IUPAC-IUBMB standards described in (Nucleic Acids Res. 13, 3021-3030, 1985 and Biochem. J., 219, 345-373, 1984) which are herein incorporated by reference. The symbols and format used for nucleotide and amino acid sequence data comply with the rules set forth in 37 C.F.R. §1.822. Tables 1 and 2 below summarize the list of sequences used in this invention. Table 3 lists trnV and rps12 homologous recombination sequences in chloroplasts from various plant species.

TABLE 1

LIST OF OLIGONUCLEOTIDE SEQUENCES USED IN THIS
INVENTION

| | | |
|---|---|---|
| SEQ ID NO | Primer Name | SEQUENCE |

| | | |
|---|---|---|
| SEQ ID NO:1 | rbc89 | CCGTGGCCACAAACAGAGACTAAAGC |
| | | AAGTGTTGG |
| SEQ ID NO:2 | rbc90 | TAGCGGCCGCTTAGTGATGGTGATGG |
| | | TGATGCTTATCCAAAACGTCCACTGC |
| | | TG |
| SEQ ID NO: 3 | rbc87 | GATCATATTCACTCTGGAACCGTAGT |
| | | AGGTAAAC |
| SEQ ID NO:4 | rbc88 | GTTTACCTACTACGGTTCCAGAGTGA |
| | | ATATGATC |
| SEQ ID NO:5 | rbc120 | TGGCGGCCGCCCCGGGCAACCCACTA |
| | | GCATA |
| SEQ ID NO:6 | rbc121 | GGGGATCCATGGTAAAATCTTGGTTT |
| | | ATTTAA |
| SEQ ID NO:7 | rbc122 | TACCATGGCACCACAAACAGAGACTA |
| | | AAGC |
| SEQ ID NO:8 | rbc123 | TCGAATTCTTAGTGATGGTGATGGTG |
| | | ATG |
| SEQ ID NO:9 | rbc124 | AAGAATTCAATTAAGGAAATAAATTA |
| SEQ ID NO:10 | rbc125 | ATAAGCTTAATTCAATGGAAGCAATG |
| | | ATA |
| SEQ ID NO:11 | rbc116 | GAGGTACCACCGCCGTATGGCTGACC |
| | | GGC |
| SEQ ID NO:12 | rbc117 | GCCTCGAGTTGACAATTGAATCCGAT |
| | | TTTG |
| SEQ ID NO:13 | rbc118 | GAGGTCGACCTGCCCCTATCGGAAAT |
| | | AGGA |
| SEQ ID NO:14 | rbc119 | TCAAGCTTTTCTTGATCAATCCCTTT |
| | | GCC |
| SEQ ID NO:15 | rbc146 | GTTACGACTTCACTCCAGTCACTAGC |
| SEQ ID NO:16 | rbc147 | CCGAAGAGTAACTAGGACCAATTTAG |
| SEQ ID NO:17 | rbc90 | TAGCGGCCGCTTAGTGATGGTGATGG |
| | | TGATGCTTATCCAAAACGTCCACTGC |
| | | TG |
| SEQ ID NO:18 | ATrbcL | GGTGGAGGAACTTTAGGACATCC |
| SEQ ID NO:19 | Trans-Tob- | GCGAGGCTTGCAAATGGA |
| | rbcL-1337F | |
| SEQ ID NO:20 | Trans-Tob- | GTGATGGTGATGGTGATGCTTA |
| | rbcL- | |
| | 1449R | |
| SEQ ID NO:21 | Endo-Tob- | CTCGTAATGAAGGACGTGATCTTG |
| | rbcL-1289F | |
| SEQ ID NO:22 | Endo-Tob- | TTCCGGGCTCCATTTGC |
| | rbcL | |
| | 1362R | |
| SEQ ID NO:23 | Tobacco | GTCCGCATGGCCCTTATG |
| | 18S-1515F | |
| SEQ ID NO:24 | Tobacco | CCTTGCTTCCCATTGTAATTGC |
| | 18S-1580R | |
| SEQ ID NO:25 | rbc154 | ATGCATATGGACCAGTCATCTCGT |
| SEQ ID NO:26 | rbc155 | TAGAATTCTTACGCCGGAAGGGCGCT |
| | | GCG |
| SEQ ID NO:27 | rbc141 | GTTTGTGGTGCCATATGGGTAAAATC |
| | | TTGG |
| SEQ ID NO:28 | rbc142 | CCAAGATTTTACCCATATGGCACCAC |
| | | AAAC |
| SEQ ID NO:29 | rbc211 | TTCTACCTCCACGCGGCATT |
| SEQ ID NO:30 | rbc212 | GCCTGATTATCCCTAAGCCCAA |

TABLE 2
HOMOLOGOUS RECOMBINATION ARM SEQUENCES
AND TRANSGENE SEQUENCES DESIGNED
FOR USE IN THIS INVENTION

| SEQ ID NO | NAME |
|---|---|
| SEQ ID NO 31 | Tobacco chloroplast 16srDNA-trnV |
| SEQ ID NO 32 | Tobacco rps12/7 |
| SEQ ID NO 33 | cpNTrbcL transgene |
| SEQ ID NO 34 | amino acid sequence of cpNTrbcL transgene |
| SEQ ID NO 35 | RRrbcM transgene |
| SEQ ID NO 36 | amino acid sequence of RRrbcM |
| SEQ ID NO: 37 | aadA3 |
| SEQ ID NO: 38 | amino acid sequence of aadA3 |

TABLE 3
trnV and rps12 fragment sequences from various plant species
that match homologous recombination arms used in this invention.

| | | GENBANK | |
|---|---|---|---|
| SEQ ID # | NAME | DESIGNATION | PLANT |
| 39 | trnV | DQ386163GP | Solanum tuberosum |
| 40 | rps12 | DQ386163gp | Solanum tuberosum |
| 41 | trnV | DQ347959 | Lycopersicon |
| | | | esculentum |
| 42 | rps12 | DQ347959 | Lycopersicon |
| | | | esculentum |
| 43 | trnV | AP007232 | Lactuca sativa |
| 44 | rps12 | AP007232 | Lactuca sativa |
| 45 | trnV | AP800956 | Populus alba |
| 46 | rps12 | AP800956 | Populus alba |
| 47 | trnV | DF17947gp | Brassica |
| 48 | rps12 | DF17947gp | Brassica |
| 49 | trnV | DQ400350 | Lemna |
| 50 | rps12 | DQ400350 | Lemna |
| 51 | trnV | DQ317523 | Glycine max |
| 52 | rps12 | DQ317523 | Glycine max |
| 53 | trnV | X15901 | Oryza sativa |
| 54 | rps12 | X15901 | Oryza sativa |

### DETAILED DESCRIPTION OF THE INVENTION

The invention provides methods for the recombinant production of soluble RUBISCO enzyme in plant cells at high levels ( i.e., levels exceeding 30% of the soluble protein) in the cell. Plant cells of the invention are constructed so as to contain a genetic construct inserted in a non-RUBISCO insertion site of the chloroplast genome. Expression of the genetic construct from this site results in high level expression of the RUBISCO enzyme. Preferred chloroplast insertion loci are in the inverted repeat region of the chloroplast genome.

The invention finds utility in that modified RUBISCO enzymes may improve the yield and growth rate of the plant. However, in order to do so, they must be expressed at very high levels. Alternatively, increased expression of native RUBISCO may also improve the yield and growth rate of the plant. Effecting such an increase in crop plants such as soybean, rice, canola, and wheat would benefit the world's food supply.

In this disclosure each reference set forth herein is hereby incorporated by reference in its entirety and the following definitions and abbreviations are to be used for the interpretation of the claims and the specification:

The term “RUBISCO” means the enzyme ribulose-1,5-bisphosphate carboxylase/oxygenase (Rubisco, E. C. 4.1.1.39), as more fully described below.

“rbcL” is the abbreviation for the RUBISCO large subunit.

“rbcS” is the abbreviation for the RUBISCO small subunit.

The term “inverted repeat region” as it applies to the chloroplast genome means a portion of the chloroplast genome having inversely originated repeating nucleotide sequences.

The term “non-RUBISCO locus” as it applies to the chloroplast genome means any portion of the chloroplast genome where a native RUBISCO is not normally expressed.

The term “crop plant” means any plant grown or produced for animal or human consumption or use. A non-limiting list of crop plants includes corn, rice, soybean, tobacco, and canola.

“Heterologous” with respect to sequence means a sequence that originates from a foreign species, or, if from the same species, is substantially modified from its native form in composition and/or genomic locus by deliberate human intervention.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation. “Transgenic plant” includes reference to a plant which comprises within its genome a heterologous polynucleotide. Preferably, the heterologous polynucleotide is stably integrated within the genome such that the polynucleotide is passed on to successive generations. The heterologous polynucleotide may be integrated into the genome alone or as part of a recombinant DNA construct.

“Genome” as it applies to plant cells encompasses not only chromosomal DNA found within the nucleus, but organelle DNA found within subcellular components (e.g., mitochondrial, plastid) of the cell.

“Polynucleotide”, “nucleic acid sequence”, “nucleotide sequence”, or “nucleic acid fragment” are used interchangeably and is a polymer of ribonucleic acid (RNA) or deoxyribonucleic acid (DNA) that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

“Messenger RNA (mRNA)” refers to the RNA that is without introns and that can be translated into protein by the cell.

“cDNA” refers to a DNA that is complementary to and synthesized from an mRNA template using the enzyme reverse transcriptase. The cDNA can be single-stranded or converted into the double-stranded form using the Klenow fragment of DNA polymerase I.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

An “inverted repeat” is a sequence of nucleotides that is the reversed complement of another sequence further downstream. Inverted repeats define the boundaries in transposons. Inverted repeats also indicate regions capable of self-complementary base pairing (regions within a single sequence which can base pair with each other).

The term “amplified” means the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA) (Diagnostic Molecular Microbiology: Principles and Applications, D. H. Persing et al., Ed., Am. Soc. Microbiol., Washington, D.C., 1993). The product of amplification is termed an amplicon.

“Plastid” refers to any of several pigmented or unpigmented cytoplasmic organelles such as chloroplasts, amyloplasts, leucoplasts, proplastids, and etioplasts, found in plant cells and other organisms, having various physiological functions, such as the synthesis and storage of food. All plastids are developmentally related to each other and all contain a plastome.

“Plastome” is the circular plastid genome of higher plants. It is approximately 150 kb in size and it encodes about 120 products.

The term “Transplastomic” means plants which have stably integrated into their plastome at least one expression cassette which is functional in plastids.

The term “Chloroplast” means a chlorophyll-containing plastid found in algal and green plant cells and includes all developmental stages of a chloroplast, such as proplastids, etioplasts, and mature chloroplasts. Chloroplasts and other plastids from all lower and higher plants are very similar in properties, and the present invention is therefore directed to all such organisms and their chloroplasts and plastids. In the practice of this invention, it is preferred to transform chloroplasts.

The term “Chaperonin” means protein complexes that assist the folding of nascent, native or non-native polypeptides into their fully-assembled, functional state.

The term “Primer” means a nucleic acid strand (or related molecule) that serves as a starting point for DNA replication.

“PCR” means polymerase chain reaction.

“Quantitative Polymerase chain reaction (qPCR) is a modification of the PCR used to rapidly measure the quantity of DNA, complementary DNA or RNA present in a sample.

“Oligo or oligonucleotide” refer to short sequences of nucleotides (RNA or DNA), typically with twenty or more bases.

“Gene” or “genetic construct” refers to a nucleic acid fragment that expresses a specific protein, optionally including regulatory sequences preceding (5′ non-coding sequences) and following (3′ non-coding sequences) the coding sequence. “Native gene” or “wild type gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. The term “open reading frame” refers to that portion of a gene or genetic construct that encodes a polypeptide but may be devoid of any regulatory elements.

A “terminator, or transcription terminator” is a section of genetic sequence that marks the end of gene or operon on genomic DNA for transcription.

“Polylinker region” means a DNA fragment on a vector/plasmid, containing multiple unique restriction enzyme recognition sites for other DNA fragments to be cloned/integrated into vector/plasmid conveniently.

“Foreign protein” means a heterologous protein.

“dNTP” is a mixture of dATP, dGTP, dCTP, and dTTP.

“GFP” means green fluorescent protein.

“NaEPPS buffer” is sodium [4-(2-hydroxyethyl)-1-piperazine-propanesulfonate buffer.

“rDNA” refers to Ribosomal Deoxyribonucleic Acid.

“Plasmid”, “vector” and “cassette” refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

“Plant” refers to any higher or lower plant, particularly dicots and monocots. In particular, the present invention is directed to plants such as lettuce (Lactuca), cabbage (Brassica), cauliflower (Brassica), potato (Solanum), rice (Oryza), soybean (Glycine), tomato (Lycopersicum), poplar (Populus), duckweed (Lemna) tobacco (Nicotiana), wheat and canola. “Progeny” comprises any subsequent generation of a plant.

“Tobacco rbcL-knockout plant”, also referred to as “rbcL-KO”, refers to a plant in which the naturally-occurring rbcL gene in the tobacco chloroplast genome is disrupted, leading to the functional inactivation of the endogenous RUBISCO. In this invention, the rbcL-KO tobacco was developed by Icon Genetics (Halle, Germany). In the chloroplast genome of this plant, the majority of the rbcL coding sequence was replaced with a GFP gene. The result created an rbcL fragment that encoded the N-terminal 59 amino acids translationally fused with the green fluorescent protein (GFP) gene. Thus, there is no functional rbcL gene in the genome and the plant has no LSU accumulation. In the absence of LSU, the SSU also does not accumulate, possibly because it is proteolytically degraded due to the absence of LSU. Thus, in the rbcL-KO tobacco there is no LSU or SSU protein, no RUBISCO activity, and no photosynthesis activity. The homoplastomic rbcL knockout plant is pale and only survives when sugar is provided. Chimeric plants containing both mutant and WT sectors are able to grow slowly without sugar supplement since the WT sectors of leaves have wild type rbcL genes and being photosynthetically competent, are able to feed the non-photosynthetic mutant sections.

In the practice of the present invention, either tobacco or bacterial DNA is provided for transformation into a plant chloroplast. “Foreign” or “exogenous” DNA refers to any DNA which is not found within the tobacco chloroplast in nature or modified from a native one. Thus, foreign DNA can encompass a wide variety of DNA molecules. Particularly preferred are DNA molecules containing an expression cassette; i.e., a DNA construct comprising a coding sequence and appropriate control sequences (e.g., promoter and appropriately matched transcription termination sequence) to provide for the proper expression of the coding sequence in the chloroplast. Typically, the expression cassette is flanked by convenient restriction sites to facilitate cloning. In a preferred embodiment, the foreign DNA used for transformation comprises an expression cassette flanked by chloroplast DNA to facilitate the stable integration of the expression cassette into the chloroplast genome by homologous recombination.

“Homologous targeting sequences” are fragments of chloroplast genome sequences, flanking the chimeric transgene structure in the plasmid. They function to exchange the chimeric transgene structure into the chloroplast genome to replace the genomic sequence between the homologous targeting sequences through homologous recombination. Homologous targeting sequence on one side is left targeting region (LTR) and on other side is right targeting region (RTR). These sequences are also referred to as “homology arms”. The term “homology arm” therefore refers to a nucleotide sequence which enables homologous recombination between two nucleic acids having substantially the same nucleotide sequence in a particular region of two different nucleic acids. The preferred size range of the nucleotide sequence of the “homology arm” is from about 300 to about 2500 nucleotides. “Homology arms” of this invention are typically found in vectors carrying the genetic construct encoding RUBISCO enzyme and are designed with homology to various regions of the chloroplast genome, preferably the inverted repeat region.

“Homologous recombination” (or general recombination) is defined as the exchange of homologous segments anywhere along a length of two DNA molecules. An essential feature of general recombination is that the enzymes responsible for the recombination event can presumably use any pair of homologous sequences as substrates, although some types of sequence may be favored over others.

The term “sequence analysis software” refers to any computer algorithm or software program that is useful for the analysis of nucleotide or amino acid sequences. Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp, CABIOS. 5,151-153, 1989) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Unless otherwise stated, “BLAST” sequence identity/similarity values provided herein refer to the value obtained using the BLAST 2.0 suite of programs using default parameters (Altschul, et al., J. Mol. Biol. 215, 403-410, 1990). Software for performing BLAST analyses is publicly available, e.g., through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=⁻4, and a comparison of both strands. For amino acid sequences, the BLASTP program uses as defaults a wordlength (W) of 3, an expectation (E) of 10, and the BLOSUM62 scoring matrix (Henikoff, S., and Henikoff, J. G., Proc. Natl. Acad. Sci. USA 89, 10915-10919, 1989).

The term “holoenzyme” in this context means an active, complex enzyme consisting of all subunits.

The terms “k_{cat}” and “Kₘ” are known to those skilled in the art and are described in Enzyme Structure and Mechanism, 2^{nd} ed. (Ferst; W. H. Freeman: N.Y., pp 98-120, 1985). The term “k_{cat}”, often called the “turnover number”, is defined as the maximum number of substrate molecules converted to products per active site per unit time, or the number of times the enzyme turns over per unit time. k_{cat}=Vmax/[E], where [E] is the enzyme concentration (Ferst, supra). The terms “total turnover” and “total turnover number” are used herein to refer to the amount of product formed by the reaction of a RUBISCO enzyme with substrate.

The term “specific activity” means enzyme units/mg protein where an enzyme unit is defined as moles of product formed/minute under specified conditions of temperature, pH, [S], etc.

RUBISCO “specificity”, sometimes designated as “Tau” or “S_{c/o}” is a measure of the rates of carboxylation to oxygenation at equal concentrations of CO₂ and O₂. It is defined by the expression:

(k^{c}_{cat}×Kₒ)/(k^{o}_{cat}×K_{c}) where:
k^{c}_{cat} is the turnover number for carboxylation,
k^{o}_{cat} is the turnover number for oxygenation
K_{c} is the Michaelis constant for CO₂
Kₒ is the Michaelis constant for O₂

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

The present invention relates to plants comprising a chloroplast genome having inserted therein a heterologous genetic construct encoding a RUBISCO enzyme, wherein the genetic construct is inserted at a non-RUBISCO locus in the genome and wherein the RUBISCO enzyme is selected from the group consisting of: a RUBISCO large subunit and a RUBISCO small subunit. Additionally the invention relates to methods for the expression of a RUBISCO enzyme in a plant comprising:

a) Providing a plant comprising a chloroplast genome;

b) Providing a vector consisting essentially of the general structure:

HA1-hetero Pro1::M::Ter1 hetero Pro2::RBC::Ter2-HA2

Wherein:
i) hetero Pro1 is a first promoter derived from a non-RUBISCO plant gene;
ii) M is a genetic construct encoding a selectable marker;
iii) Ter1 is a first terminator;
iv) hetero Pro2 is a second promoter derived from a non-RUBISCO plant gene;
v) RBC is a genetic construct encoding a plant protein selected from the group consisting of: the small subunit of a RUBISCO enzyme and the large subunit of an RUBISCO enzyme;
vi) Ter2 is a second terminator;
vii) HA1 is a first homology arm having homology to a first section of the inverted repeat region of the chloroplast genome; and
viii) HA2 is a second homology arm having homology to a second section of the inverted repeat region of the chloroplast genome; and

c) transforming the plant of (a) with the vector of (b) wherein the vector inserts in the inverted repeat region of the chloroplast genome and the RBC is a genetic construct which is expressed producing a RUBISCO enzyme in soluble form.

### RUBISCO Enzyme

Three major forms of RUBISCO enzymes are found in living organisms (Andrews T. J., & Lorimer, G. H., The Biochemistry of Plants, volume 10, 131-218, 1987 and Miziorko, H. M., & Lorimer, G. H., Annu. Rev. Biochem., 52, 507-535, 1983). Form I, which is found in higher plants, algae and most other photosynthetic organisms, is a complex molecule consisting of eight large (L, Mr=55, 000) and eight small (S, Mr=14,000) subunits, forming an L₈S₈ complex. On the other hand, form II, which is primarily found in certain bacteria, e.g., the photosynthetic bacterium Rhodospirillum rubrum (R. rubrum), is a dimer of large subunits, L₂, (Tabita, F. R. and McFadden, B, A., Arch. Microbiol., 99, 231-40, 1974) that differ substantially in sequence from form I large subunits. Depending on the source, form II may be oligomerized to form dimers, tetramers, or even larger oligomers (Li, H., et al., Structure, 13, 779-789, 2005). Form III also contains only a LSU and forms dimers (L₂) or decamers [(L₂)₅] (Li, H., supra). In all forms the L subunit carries the catalytic function of the enzyme.

In higher plants, the LSU subunit of the form I RUBISCO is encoded by the chloroplast gene rbcL while the SSU subunit is encoded by the nuclear gene rbcS. After synthesis, SSU is translocated from the cytosol to the chloroplast, processed to remove the transit peptide, and assembled with the LSU subunit. The prokaryotic form II RUBISCO, e.g., the one present in R. rubrum , has two LSU, encoded by a single rbcM gene (also known as cbbM). The gene for the L subunit of R. rubrum RUBISCO has been cloned and expressed in E. coli (Somerville, C. R. and Somerville, S. C., Recherche, 15, 490-501, 1984 and Pierce, J. and Gutteridge, S., Appl. Environ. Microbiol., 49, 1094-100, 1985) and shown to be a fusion protein consisting of RUBISCO and 24 additional amino acids from β-galactosidase at the N-terminus. The catalytic and kinetic properties of the fusion protein were indistinguishable from the wild-type enzyme.

The RUBISCO enzyme from plants is a sub-optimal enzyme in two respects. First, its catalytic activity (k^{c}_{cat}˜3s⁻¹), is relatively slow for an enzyme that performs such a high flux reaction in photosynthetic carbon fixation. To compensate for its low activity, plants accumulate large amounts of RUBISCO enzyme in their green tissues. Indeed, RUBISCO accounts for about half of the leaf's total soluble proteins (TSP). Increasing RUBISCO's catalytic rate, therefore, would commensurately reduce the requirement for the massive accumulation of enzyme and allow the plant to reapportion catalytic resources to other functions. Second, RUBISCO has poor ability to discriminate between CO₂ and O₂, leading to catalysis of both carboxylation and oxygenation of RuBP. The ability of RUBISCO to discriminate between CO₂ and O₂ is measured by the specificity (also known as selectivity or Tao, τ, or S_{c/o}). The specificity represents the ratio of the rates of carboxylation to oxygenation at equal concentrations of CO₂ and O₂ and is expressed as [(k^{c}_{cat})(Kₒ)]/[(k^{o}_{cat})(K_{c})] (Laing et al., Plant Physiol., 54, 678-685, 1974). The RUBISCO from higher plants typically has a specificity of 80-100, however, because of the much higher concentration of O₂ than CO₂ in the atmosphere, about 25% of the turnovers of RUBISCO in most plants are oxygenations.

Compared to plant RUBISCO, the enzyme from prokaryotic photosynthetic bacteria generally possesses higher catalytic activity (k_{cat}8-16 s⁻¹), but low CO₂/O₂ selectivity (τ≈13-40). The RUBISCO from red algae shows the highest CO₂/O₂ selectivity yet measured (τ≈140-300, (Ezaki, S., et al., J. Biol. Chem., 274, 5078-5082, 1999 and Read, B. A. and Tabita, F. R., Arch. Biochem. Biophys. 312, 210-218, 1994, and Uemura, K., et al., Biochem. Biophys. Res. Commun. 233, 568-571, 1997). Models which relate RUBISCO parameters to photosynthesis, growth, and yield have been developed (von Caemmerer, S., Biochemical Models of Leaf Photosynthesis 2000, CSIRO Publishing and Zhu, X. et al., Plant Cell and Environment, 27, 155-165, 2004 and Alagarswamy, G., et al. Agron. J., 98, 34-42, 2006 and Whitney, S. M. and Andrews, T. J., Plant Physiol., 133, 287-294, 2003). These models predict that increasing RUBISCO's catalytic efficiency will result in a substantial increase in plants' productivity. In particular, if the oxygenase activity were eliminated and the rate of carboxylation increased about ten-fold, plant productivity would be predicted to increase by 50%. There has therefore been a long felt need for production of a superior RUBISCO enzyme in higher plants to improve the efficiency and rate of photosynthesis (Spreitzer, R. J. and Salvucci, M. E., Ann. Rev. Plant Biol., 53, 449-475, 2002 and Whitney, S. M. and Andrews, T. J. supra and Parry, M. A. J., et al., J. Exp. Botany, 54, 1321-1333, 2003 and Mann, C. C., Science, 283, 314-316, 1999, and references cited therein).

Development of a method for transformation of plant chloroplasts with an active RUBISCO enzyme having improved kinetic properties will be useful to improve photosynthesis for better crop performance and is therefore important. In order to be agronomically useful, such a method must express the new RUBISCO at substantial levels. For example, a RUBISCO with a k^{c}_{cat} equivalent to the plant enzyme (˜3 s⁻¹) will need to be expressed at approximately the same levels as the endogenous RUBISCO, or about 50% of the soluble leaf protein. For enzymes with higher k^{c}_{cat} values, the requisite expression level will be somewhat lower. Expression of any foreign protein at this level in plants is extremely difficult. For example, the highest expression from nuclear transgenes is about 10% of TSP (Outchkourov, N. S., et al., Planta, 216, 1003-1012, 2003). While a heterologous functional expression and accumulation of up to 25% of TSP of a thermostable endo-1,4-beta-D-glucanase in Arabidopsis leaves, using an apoplast targeting approach, has been reported (Ziegler, M. T., et al., Mol Breeding, 6:37-46, 2000), todate such heterologous expression levels in plants are usually low and only around 0.01-1.0% (Franken, E. et al., Curr. Op. Biotechnol., 8, 411-416, 1997). Heterologous protein expression in tobacco chloroplasts have produced the desired protein at levels of around 10-25% of the TSP (Khan, M. S. and Maliga, P., Nature Biotechnol.,17, 910-915, 1999 and Kuroda, H. and Maliga, P., Plant Physiol., 125, 430-436, 2001 and Kuroda, H. and Maliga, P. Nuc. Acid Res., 29, 970-975, 2001b and Maliga, P., Trends in Biotechnol., 21, 20-28, 2003). Co-expression of a special chaperonin with Bacillus thuringiensis (Bt) protein increased its production up to 45% of the TSP (De Cosa, B., et al., Nature Biotechnol., 19, 71-74, 2001).

High level functional expression of the rbcL genes of RUBISCO into the tobacco plastome have been achieved. In one study the sunflower (Helianthus annus) rbcL gene introduced into the tobacco plastome between the atpB and accD genes produced functional sunflower RUBISCO LSU at approximately 15% of the TSP (Kanevski, I., et al., Plant Physiol., 119, 133-141, 1999). In another study the majority of the tobacco LSU coding region (from the 15^{th} codon to the stop codon) was replaced by R. rubrum rbcM introduced into its plastome. (Whitney, S. M., et al., Plant J., 26, 535-547, 2001). The R. rubrum RUBISCO LSU produced by the transplastomic plant was fused to the first 14 residues of the tobacco RUBISCO LSU and accumulated to approximately 15% of the TSP. This enzyme showed typical R. rubrum RUBISCO kinetic properties. In another study a C-terminally His-tagged tobacco RUBISCO LSU was made and transformed into the WT tobacco to replace the endogenous LSU gene. The resulting transformants produced a functional, soluble His-tagged RUBISCO at wild type levels (Rumeau, D. et al., Plant Biotechnol. J., 2, 389-399, 2004).

### Plant Transformation

The present invention relates to the expression of a RUBISCO enzyme from a plant chloroplast genome. Preferred plants for this expression are crop plants where tobacco, rice, wheat soybean and canola are particularly suitable. Transgenic plant cells are placed in an appropriate selective medium for selection of transgenic cells that are then grown to callus. Shoots are grown from callus and plantlets are generated from the shoot by growing in a rooting medium. Various constructs will normally be joined to a marker for selection in the plant cells. Conveniently, the marker may be resistance to a biocide (particularly an antibiotic such as kanamycin, G418, bleomycin, hygromycin, chloramphenicol, herbicide, spectinomycin or the like). The particular marker used will allow for selection of transformed cells as compared to cells lacking the DNA that has been introduced. Components of DNA constructs including transcription cassettes of this invention may be prepared from sequences which are native (endogenous) or foreign (exogenous) to the host. By “foreign” it is meant that the sequence is not found in the wild-type host into which the construct is introduced. Heterologous constructs will contain at least one region that is not native to the gene from which the transcription initiation region is derived.

To confirm the presence of the transgenes in transgenic cells and plants, a Southern blot or PCR analysis can be performed using methods known to those skilled in the art. Expression products of the transgenes can be detected by various methods (e.g., western blot and enzyme assay), depending upon the nature of the product. Once transgenic plants have been obtained, they may be grown to produce plant tissues or parts having the desired phenotype. The plant tissue or plant parts may be harvested, and/or the seed collected. The seed may serve as a source for growing additional plants with tissues or parts having the desired characteristics.

A desirable approach for expressing foreign RUBISCO genes is to introduce the gene into the genome of the chloroplast, the organelle in which the rbcL gene normally resides. Methods have been disclosed for introducing genes into the chloroplast genome. Chloroplast transformation vectors use regulatory and untranslated regions (promoters, ribosome binding sites and terminators) of chloroplast origin to control expression of marker genes and specific genes of interest (such as RUBISCO genes). These genes are flanked by chloroplast sequences (homologous targeting sequences or homology arms) that are homologous to specific sites in the chloroplast genome. Following delivery of the chloroplast transformation vector into a chloroplast, these homologous targeting sequences mediate homologous recombination between the introduced vector and the chloroplast genome, resulting in the insertion of the sequence in the vector located between the homologous targeting sequences into the chloroplast genome. The introduced genes are then expressed as soluble proteins at high levels (at least about 30% of the TSPs).

Methods disclosed for plastid transformation in higher plants include the particle gun delivery of DNA containing a selectable marker and targeting of the DNA to the plastid genome through homologous recombination (Svab, Z. et al., Proc. Natl. Acad. Sci., USA, 87, 8526-8530, 1990 and Svab and Maliga, Proc. Natl. Acad. Sci., USA, 90, 913-917, 1993 and Staub and Maliga, EMBO J., 12, 601-606, 1993 and U.S. Pat. Nos. 5,451,513 and 5,545,818). In some species, protoplasts can also be used for chloroplast transformation (O'Neill, C., et al., Plant J., 3, 729-38, 1993 and Spoerlein, B., et al., Theor. Appl. Gen., 82, 717-722, 1991). Following introduction of the chloroplast transformation vectors, the treated cultures are placed on a tissue culture medium containing the appropriate selection agent.

The most commonly used selection marker is the aadA gene coding for streptomycin/spectinomycin adenyltransferase (Svab, Z. et al., Proc. Natl. Acad. Sci., USA, 90, 913-917, 1993). Genes conferring resistance to kanamycin (NPTII or AphA6) have also been used (Carrer, H., et al., Mol. Gen. Genetics, 241, 49-56, 1993 and Huang, F.-C. et al., Mol. Gen. Genomics, 268, 19-27, 2002). After a suitable period of incubation on selection medium, transformed cell lines can be identified and grown to a stage that allows regeneration of the whole plants. The regeneration processes are basically identical to those used for standard nuclear transformation events. Special care must be taken to ensure that selection and regeneration conditions promote the elimination of all wild-type chloroplast genomes. The status of the proportion of wild-type to transformed chloroplast genomes can be monitored by standard molecular techniques including Southern and PCR analysis.

The chloroplast genomes of higher plants are fairly well conserved and the rbcL gene is usually found with the same genetic context in a wide variety of species. Successful introduction of the rbcL gene for production of functional soluble RUBISCO holoenzyme in the past has been possible through insertion of an rbcL gene at the native rbcL site. In these cases the introduced gene was under the control of the native rbcL promoter and other rbcL regulatory elements, thus ensuring that expression of the introduced rbcL gene is regulated correctly to meet the requirements for the plant to be able to utlize it. In this invention, the introduced gene has been inserted at a site at least 2 kb away from the location of the native rbcL gene. This location has been defined as a “non-rbcL” or “non-RUBISCO” site. Also in the present invention, expression of the introduced rbcL coding sequence has been controlled by a promoter and other regulatory elements that are not normally used by the native rbcL gene. These elements have been identified as “non-rbcL regulatory elements”. Use of these elements offer the potential to confer different expression properties on the introduced RUBISCO genes, thus creating the possibility of generating altered and potentially improved expression patterns. On the other hand, these non-rbcL regulatory elements may well confer unacceptable expression patterns that prevent the introduced RUBISCO from functioning effectively in the plant.

Whitney, et al. (Plant J., 26, 535-547, 2001) have described introduction of RUBISCO genes from two organisms, the diatom Phaeodactylum tricornutum and the rhodophyte Galdieria sulphuraria at a non-rbcL site in the tobacco chloroplast genome. However in both of these cases, the introduced genes did not produce functional, soluble RUBISCO holoenzyme.

Inverted repeats are desirable sites because introduction of transgenes into the inverted repeat region results in the presence of two copies of the introduced gene per chloroplast genome as opposed to just one copy for insertions outside the inverted repeat regions. Doubling of the copy number of the gene could yield higher levels of gene expression compared to transgenes introduced into single copy regions of the chloroplast genome. Since the regions between trnV and rps12 loci (see Table 3—SEQ ID Nos 39-54) in the inverted repeats are transcriptionally silent the transgenes being inserted in these regions are therefore less likely to interfere with or be interfered by the surrounding genes. The present invention provides the first example of a functional RUBISCO gene expressed from a location in the chloroplast genome other than the native rbcL site where the rbcL gene normally resides.

The assembly of the L8S8 RUBISCO holoenzyme from its constituent subunits is a complex and poorly understood process and does not proceed properly in heterologous systems. For example, there are no demonstrated examples of L₈S₈ enzymes from prokaryotic or eukaryotic algae assembling into functional, soluble holoenzymes in plants (e.g. Whitney, et al., supra and Kanevski I, et al., Plant Physiol 119, 133-141, 1999). In addition, proper assembly of a plant enzyme in a non-plant host has not been previously reported. In many cases in both plants and prokaryotes, the lack of proper assembly of the holoenzyme leads to the accumulation of RUBISCO protein as insoluble, non-functional aggregated proteins (e.g. Whitney, et al. supra). An exceptional case was reported by Kanevski et al., and Sharwood et al. (Kanevski et al., supra, and Sharwood et al., Plant Physiol., 146, 83-96, 2008) when they successfully assembled the higher plant sunflower's rbcL with tobacco's rbcS to form a functional holoenzyme.

In the present invention, a novel vector is used to express RUBISCO genes in the chloroplast genome. This novel vector targets the gene insertion to a non-RUBISCO locus using non-RUBISCO regulatory elements. Neither of these attributes has previously been used for successful expression of functional, soluble RUBISCO holoenzmes in plants.

Chloroplast transformation has been accomplished in a number of plants including tobacco, soybean, rice, Brassica, potato, soybean, duckweed, lettuce, cabbage, tomato, cotton, and poplar (Li, Yi-Nu et al., Zhongguo Nongye Kexue, Beijing, China, 40, 1849-1851, 2007, and Hou, Bingkai; et al., 28, 187-192, 2002, and Nguyen, T., et al, Plant Sci., 168, 1495-1500, 2005, and Dufourmantel, N., et al., Plant Mol. Biol., 55, 479-489, 2004, and Cox, K. M., and Peele, C. G. PCT Int. Appl., 2005, WO 2005005643 A2 20050120, and Kanamoto, H., et al., Transgenic Res., 15, 205-217, 2006, and Liu, Cheng-Wei, et al., Plant Cell Rep., 26, 1733-1744, 2007, and Wurbs, D., et al., Plant J., 49, 276-288; 2007, and Kumar, S., et al., Plant Mol. Biol., 56, 203-216, 2004, and Okumura, S., et al., Transgenic Res., 15, 637-646, 2006 and Svab, Z., et al., Proc. Natl. Acad. Sci. USA, 87, 8526-8530, 1990 and WO2004053133A1 and US20070039075A1).

Chloroplast transformation has been used to introduce foreign RUBISCO genes into the chloroplast genome. For example, a C-terminally His-tagged tobacco RUBISCO rbcL (D. Rumeau, et al., Plant Biotechnol. J., 2, 389-399, 2004), the rbcL gene from sunflower (Kanevski, I., et al., supra) and the rbcM gene from the bacterium R. rubrum (Whitney, S. M. and Andrews, T. J. Plant Physiol., 133, 287-294, 2003 and Whitney, S. M. and Andrews, T. J., Proc. Natl. Acad. Sci., USA, 98, 14738-14743, 2001) were introduced into the chloroplast genome of tobacco. The rbcL and rbcS genes from the red alga Galdieria sulphuraria and the diatom Phaeodactylum tricornutum were also introduced into the chloroplast genome of tobacco (Whitney, S. M., et al., Plant J., 26, 535-547, 2001). Large amounts of RUBISCO protein was expressed from all these transgenes, however those from the red alga and the diatom were not properly assembled into a functional holoenzyme.

The psbA promoter from the plant chloroplast has been used in a number of studies to express foreign proteins to high levels. For example, Hayashi and coworkers (Plant Cell Physiol., 44, 334-341, 2003) used the psbA promoter to drive expression of the green fluorescent protein in tobacco chloroplasts. The vector used the following elements where T before a component designates a terminator and P designates a promoter: TpsbA::aadA::Prrn//PpsbA::gfp::Trps16. This construct was introduced between the trnV and rps12/7 genes of the chloroplast genome. Staub and coworkers (Nature Biotechnol., 18, 333-338, 2000) used the psbA promoter to express human somatotropin (hST). They used the following vector with the components: PpsbA::hST::Trps16//Prr::aadA::Trps16. The transgenes were inserted between the trnV and rps12/7 genes of the chloroplast genome. hST production was 7% of TSP. Dhingra and coworkers (Proc. Natl. Acad. Sci., USA, 101, 6315-6320, 2004) used the psbA promoter to control expression of the tobacco rbcS gene. The components of the vector were: Prrn::aadA/PpsbA::rbcS::TpsbA. This construct was inserted between the trnI and trnA genes and gave 106% of the wild type rbcS levels. Dufourmantel and coworkers (Plant Biotechnol. J., 5, 118-133, 2007) used the psbA promoter to drive expression of HPPD (4-hydroxyphenylpyruvate dioxygenase). The construct was inserted between the rbcL and acetyl CoA-carboxylase (accD) genes in tobacco and lead to the accumulation of the foreign protein at 5% of TSP. The structure of the vector that was used was:
PpsbA::HPPD::TrbcL//Prrn::aadA::TpsbA.

The psbA promoter and a host of other promoters, 5′ UTRs, terminators and homology regions have been used to express a wide range of proteins. These applications are summarized in the following review articles: (Daniell, H., et al., Transgenic Plants, 83-110, 2003, and Bock, R., Cur. Op. Biotechnol., 18, 100-106, 2007, and Grevich, J. and Daniell, H., Crit. Rev. Plant Sci, 24, 83-107, 2005, and Maliga, P., Ann. Rev. Plant Biol., 55, 289-313, 2004).

In one embodiment, an extremely efficient method was developed for functional expression and accumulation of a transgenic tobacco RUBISCO LSU protein at 50% or higher levels of the TSP in tobacco leaf. A master chloroplast expression plasmid pTCP101 was first created and then the marker gene structure NTPrrn::aadA3::TpsbA and transgene structure PpsbA::cpNTrbcL::Trps16 were introduced into this plasmid to create the chloroplast transformation vector pTCP102. The marker gene and transgene in pTCP102 were introduced into the chloroplast genome of a rbcL-KO tobacco, between the 16SrDNA-trnV and rps12/7 regions, through homologous recombination. The two introduced genes were oriented in opposite directions, away from each other, and supported expression of both the aadA3 marker and the cpNTrbcL transgene for efficient selection of transformants and high level expression of a tobacco LSU-6His fusion protein. Expression of the cpNTrbcL transgene in the transplastomic plant at both the transcript and protein levels was greater than the expression of the endogenous rbcL in the control wild type plant.

In one embodiment it was shown that the amount of the cpNTrbcL transcript in the transformants was approximately two-fold higher than the rbcL transcript in the wild type plants.

In another embodiment, it was demonstrated that the pTCP102 plasmid supported high level expression of LSu-6His up to more than 50% TSP from the cpNTrbcL transgene following chloroplast transformation. The cpNTrbcL product interacted with the endogenous RUBISCO SSU and formed the L₈S₈ RUBISCO complex exhibiting RUBISCO activity, albeit at lower levels compared to the wild type. The lower activity was probably due to the presence of the C-terminal 6-His tag in LSU-6His.

In another embodiment the cpNTrbcL in pTCP102 was replaced by the prokaryotic R. rubrum RUBISCO LSU gene RRrbcM, forming the pTCP107 plasmid. Transformation of pTCP107 into the chloroplasts of the rbcL-KO tobacco, resulted in the high level expression (up to 41% TSP) of the R. rubrum RUBISCO protein in tobacco leaf tissue. The R. rubrum RUBISCO LSU has only a 30.5% identity to tobacco RUBISCO LSU. An L₂ RUBISCO complex, with high RUBISCO activity, was observed in the transplastomic plant.

The pTCP101-derived expression vector therefore, supported functional high-level expression of both form I and form II RUBISCO genes in tobacco chloroplasts. Since R. rubrum RUBISCO has a primary sequence and holoenzyme complex distinct from tobacco RUBISCO, this embodiment strongly reinforces the potential of pTCP101-derived plasmids to support functional high level expression of other heterologous proteins in chloroplasts.

An aspect of the invention provides tobacco plants containing a polynucleotide sequence encoding either the tobacco RUBISCO large subunit or a prokaryotic R. rubrum RUBISCO gene contained in an expression cassette suitable for expression in tobacco chloroplasts. Optionally, an additional expression cassette encoding a complementing RUBISCO small subunit operably linked to regulatory sequences for expression in the chloroplast of the tobacco plant is introduced into the tobacco chloroplast. The large and small subunit expression cassettes are introduced into the chloroplasts of a regenerable plant cell (e.g. in an intact leaf or an isolated protoplast of a plant cell) by transformation methods well known in the art.

Also optionally, an additional expression cassette encoding a complementing RUBISCO small subunit operably linked to nuclear regulatory sequences for expression from a nuclear gene followed by synthesis in the cytoplasm and transport to the chloroplast tobacco plant is introduced into the tobacco nucleus. The large subunit expression cassette is introduced into the chloroplasts of a regenerable plant cell (e.g. a protoplast of a plant cell), and optionally the small subunit expression vector is introduced into the nucleus of the regenerable plant cell, both by transformation methods well known in the art.

Also optionally, the expression of the endogenous SSU genes is reduced by introducing into the nucleus constructs that suppress expression of these genes by antisense (Rodermel, S. R., et al., Cell 55, 673-681, 1988) or co-suppression approaches or by other mutational means by methods well known in the art.

### RUBISCO Enzyme Assay

Plant samples, in which the level of RUBISCO enzyme was to be determined, were powdered under liquid nitrogen and stored at −80° C. until required. Samples of ˜20 mg fresh weight (FW) were extracted by vigorous shaking with 1.0 ml of extraction buffer, leading to an initial ˜50-fold (w/v) dilution. The composition of the extraction buffer was 20% (v/v) glycerol, 0.25% (w/v) bovine serum albumin,1% (v/v) Triton-X100 (Sigma), 50 mM 4-(2-hydroxyethyl)-1-piperazineethanesulfonic acid (HEPES)/KOH pH 7.5, 10 mM MgCl₂, 1.0 mM ethylenediaminetetra acetic acid (EDTA), 1.0 mM ethylene glycolbis(beta-aminoethylether)-N,N,N′,N′-tetraacetic acid (EGTA), 1.0 mM benzamidine, 1.0 mM Me-aminocapronic acid, 1.0 mM phenylmethane-sulphonylfluoride (PMSF), 10 mM leupeptin and 0.5 mM dithiothreitol. PMSF was added just prior to extraction.

RUBISCO activity in plant extracts was determined by measuring ribulose-1,5-bisphosphate (RuBP) dependent ¹⁴CO₂ fixation. The RUBISCO was first activated by addition of 20 mM each of MgCl₂ and NaHCO₃ followed by incubation at room temperature for one h.

Reactions were performed in 30 μl total volume in 1.5 ml polypropylene tubes. The mixture consisted of 15 μl extract (diluted as needed with 0.1 M NaEPPS, pH 8, containing 20 mM MgCl₂, 20 mM NaHCO₃, 1.0 mM EDTA, 50 μg/ml bovine serum albumin, and 2 mM dithiothreitol). A solution of [¹⁴C]-NaHCO₃ (10 μl, 0.3 mM, in 0.1 M Na EPPS, pH8) was added. Reaction at 25° C. was started by addition of 5.0 μl of 6 mM RuBP. Three assays containing different levels of highly active extracts (to check for linearity with extract) were performed for 10 min, after which 25 μl of the reaction was transferred to a 7 ml glass vial containing 0.4 ml 10% v/v acetic acid. Two pairs of reactions were performed for less active samples. Each reaction containing RuBP was paired with another lacking RuBP, and reactions were terminated at 10 and 60 min. Three controls, each with excess enzyme for determination of the specific radioactivity of ¹⁴C in the assay and three other controls, each with no enzyme, were performed with each set of assays. The vials containing quenched reactions were taken to dryness on a hotplate (temperature 40-75° C., depending on whether slow or fast drying was desired), and taken up in 0.2 ml water. Scintillation fluid (5.0 ml, Ecolume, from MP Biologicals, Solon, Ohio) was added, and the samples were capped and counted in a Beckman LS6000TA liquid scintillation counter.

The specific activity of the ¹⁴C in the assay was calculated by subtracting the mean of the no-enzyme controls from the excess enzyme controls, averaging the result, and dividing by 25 nmol RuBP added to the aliquot collected. The RUBISCO activity in leaf samples was calculated in two ways. For active samples, the no-enzyme value was subtracted from the observed counts, and the corrected value converted to nmol ¹⁴C fixed. For less-active samples, the counts in the −RuBP reaction of each pair was subtracted from the corresponding +RuBP reaction. If the difference was considered meaningful (at least 50% higher +RuBP, in both samples), nmol ¹⁴C fixed were calculated as above. The results were then converted to μmoles/min/mg of protein taking into account the volume of extract in each assay.

It is understood, as those skilled in the art will appreciate, that the invention encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

### EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

In addition to the techniques described above, the practice of the present invention will employ conventional techniques of molecular biology, microbiology, recombinant DNA technology, and plant science, all of which are within the skill of the art. Such techniques are explained fully in the literature (Maniatis et al., Molecular Cloning: A Laboratory Manual (1982); and DNA Cloning: Volumes I and II (Glover, D. N., ed. 1985), and Oligonucleotide Synthesis (M. J. Gait ed. 1984), and Nucleic Acid Hybridization (Hames, B. D., & Higgins, S. J., eds. 1985), and Transcription and Translation (Hames, B. D. & Higgins, S. J., eds. 1984), Plant Cell Culture (Dixon, R. A., ed. 1985); and Propagation of Higher Plants Through Tissue Culture (Hughes, K. W., et al. eds. 1978), and Cell Culture and Somatic Cell Genetics of Plants (Vasil. I. K., ed. 1984), and Fraley et al. (1986) CRC Critical Reviews in Plant Sciences 4:1 (hereinafter Plant Sciences), and Biotechnology in Agricultural Chemistry: ACS Symposium Series 334 (LeBaron et al. eds. 1987)).

Additional abbreviations used in this application are as follows: “hr” or “h” means hour(s), “min” means minute(s), “day” means day(s), “ml” means milliliters, “mg/ml” means milligram per milliliter, “mg/l” means milligram per liter, “L” means liters, “μl” means microliters, “mM” means millimolar, “nmoles” or “nmole” means nano mole(s), “Cm” means centimeters, “g” means gram, “g/l” means gram per liter, “μM” means micromolar, “ng” means nano grams, “μg” means micrograms, “°C” means degrees Centigrade, “bp” means base pair, “bps” means base pairs, “kd” means kilodaltons, “psi” means per square inch, “kpb” or “kb” means kilobase pair, “v/v” means volume per volume, “sec” means second, “μmoles/min/mg” means micromoles per minute per milligram, “dpm/nmol” means disintegration per minute per nanomole.

### Example 1

### Construction of Nicotiana Tabacum RUBISCO Large Subunit Chloroplast Transgene

The aim of this Example was to construct a chloroplast expression plasmid containing the rbcL gene of tobacco for chloroplast transformation of tobacco plants.

Cloning the Tobacco rbcL Coding Sequence

To clone the rbcL gene, total DNA containing both nuclear and chloroplast genomic DNA was isolated from mature leaves of greenhouse-grown Nicotiana tabacum using DNeasy Plant Mini Kit, following the kit's instructions (Qiagen, Valencia, Calif.). DNA concentration was determined by using the Nanodrop ND-1000 technique (Nanodrop technologies, Montchanin, Del.).

The chloroplast genome of Nicotiana tabacum has been completely sequenced and is available in public databases (accession # Z00044). Based on the genome sequence, a pair of PCR primers rbc89 and rbc90 (SEQ ID NO: 1 and SEQ ID NO: 2) which flank the tobacco rbcL gene were synthesized (Sigma, St. Louis, MO). The upstream primer rbc89 was designed to delete the 2 N-terminal amino acid residues, and add a 5′ MscI site at the first codon (for proline). The downstream primer rbc90 was designed to add a C-terminal 6-histidine tag with a stop codon and a NotI site immediately following the stop codon. Both primers were used to amplify a full length chloroplast rbcL gene from the total DNA in a PCR reaction. The following conditions were used for performing the PCR reaction. A 25-μl PCR reaction consisted of 100 ng tobacco total DNA, 10 pmoles rbc89 and rbc90 primers, 5 nmoles each of dNTPs, 2.5 units of Pfu ultra enzyme and 2.5 μl Pfu ultra buffer (Stratagene, La Jolla, Calif.). The reaction was pre-heated at 95° C. for 4 min, followed by 30 cycles of denaturing at 95° C. for 1.0 min, annealing at 56° C. for 1.0 min, and extending at 72° C. for 2 min. The product was purified using QIAquick PCR Purification Kit (Qiagen) and cloned into a PCR Blunt II TOPO vector using Zero Blunt TOPO PCR Cloning Kit (Invitrogen, Carlsbad, Calif.) which resulted in pTP-NTrbcL. The inserted PCR product NTrbcL included a 1446 bp ORF encoding a 475-amino acid (aa) RUBISCO large subunit (LSU) and a 6-histidine C-terminal tag with a stop codon. The first two amino acid residues (Met and Ser) of tobacco RUBISCO LSU were not included. The sequence was flanked with a 5′ MscI site at the first codon (for proline) and a 3′ NotI site immediately following the stop codon.

The NTrbcL contained an internal KpnI site. Because it would interfere with later cloning, this KpnI site was mutated with Quikchange Site-Direct Mutagenesis Kit (Stratagene), following manufacturer's instruction. Two oligos, rbc87 (SEQ ID NO: 3) and rbc88 (SEQ ID NO: 4), were employed during mutagenesis to convert the KpnI site (GGTACC) to a sequence of GGAACC, without changing the encoded amino acid residues. The resulting vector was named pTP-NTrbcL-dKpnI.

The cpNTrbcL Transgene Construct for Tobacco Chloroplast Transformation

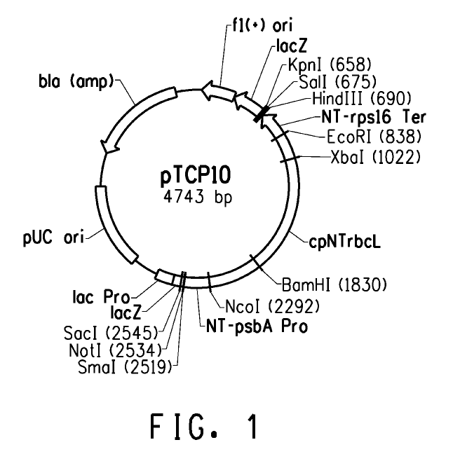

To build a chimeric cpNTrbcL transgene construct, a 227-bp psbA promoter region was amplified from the tobacco chloroplast genome in a PCR reaction, using tobacco total DNA as template as described above. The reaction used two primers: rbc120 (SEQIDNO: 5) and rbc121 (SEQ ID NO:6). These primers also introduced a BamHI site and an NotI site to the 5′ and 3′ ends of the fragment, respectively. The resulting PCR product was digested with both enzymes, purified using QIAquick PCR Purification Kit (Qiagen), and cloned into pBS SK(+) (Stratagene, LA Jolla, Calif.) between BamHI and NotI sites. The resulting plasmid was named pTCP10s1. In the second step, a cpNTrbcL fragment was amplified from pTP-NTrbcL-dKpnI using primers rbc122 (SEQ ID NO: 7) and rbc123 (SEQ ID NO: 8) in a PCR reaction as described above. These primers added a start codon and an Ala codon (ATG-GCA) to the N-terminus of NTrbcL coding sequence. The resulting PCR product had an NcoI site at the start codon and an EcoRI site immediately after the stop codon. This cpNTrbcL fragment encoded a full-length tobacco RUBISCO LSU with the second residue altered from a “Ser” to an “Ala” as compared to the wild type LSU protein and with a C-terminal 6-His tag. The PCR product was treated with NcoI and EcoRI and inserted into pTCP10s1 between NcoI and EcoRI, downstream of the psbA promoter, forming the pTCP10s2 vector. In the final step, a 148-bp rps16 terminator sequence was amplified from the tobacco chloroplast genome. Primers rbc124 (SEQ ID NO: 9) and rbc125 (SEQ ID NO: 10) were used in the PCR reaction. These primers changed the 5′ end sequence of the rps16 terminator from GAAATTC to GAATTC to create an EcoRI site, and at the 3′ end of the rps16 terminator changed the sequence from GAATTC to GAATT to remove an EcoRI site and integrate a HindIII site, respectively. The PCR fragment was digested with EcoRI and HindIII and inserted into pTCP10s2 between EcoRI and HindIII sites, downstream of the cpNTrbcL fragment. The resulting plasmid was pTCP10, containing a chimeric gene structure of psbA Pro::cpNTrbcL::rps16 Ter. A map of pTCP10 is shown in FIG. 1. DNA and amino acid sequences of cpNTrbcL are listed in Table 2 (SEQ ID NO 33 and 34).

### Expression Vector for Tobacco Chloroplast Transformation

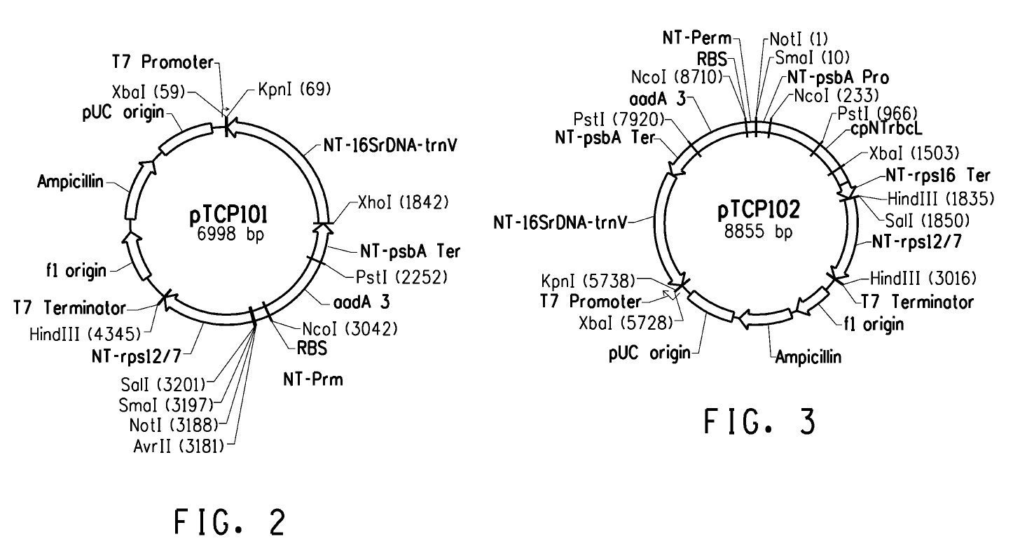

In order to establish a master expression plasmid for tobacco chloroplast transformation, a chimeric aadA3 marker gene construct containing a 117-bp tobacco Prrn promoter, a 792-bp aadA3 marker, and a 400-bp tobacco psbA terminator was designed. The aadA3 marker is a bacterial gene encoding an aminoglycoside 3′-O-nucleotidyltransferase and its sequence is available in a public database (Accession # AF047479). An optimized aadA3 coding sequence (SEQ ID NO:37 and 38) was designed according to the codon bias in higher plant chloroplasts. The three-element aadA3 chimeric gene, including the tobacco Prrn promoter, the codon optimized aadA3 coding region, and the tobacco psbA terminator was synthesized chemically and cloned into pBS SK(+) by Syngene (Bangalore, India). Then, a 1771-bp tobacco 16SrDNA-trnV sequence (SEQ ID NO 31), to serve as an arm for homologous recombination, was amplified from the tobacco chloroplast genome, using primer rbc116 (SEQ ID NO: 11) and primer rbc117 (SEQ ID NO: 12). This PCR reaction added a KpnI site and an XhoI site to the 5′ and 3′ ends of the 16SrDNA-trnV sequence, respectively. The 16SrDNA-trnV sequence was treated with KpnI and XhoI enzymes and inserted into pSnt between the KpnI and XhoI sites, downstream of the chimeric aadA3 gene to produce pTCP101s1. Finally, a 1138-bp tobacco rps12/7 sequence (SEQ ID NO 32), to serve as another arm for homologous recombination, flanked by a 5′ SalI site and a 3′ HindIII site, was amplified from the tobacco chloroplast genome, using primer rbc118 (SEQ ID NO: 13) and primer rbc119 (SEQ ID NO: 14). The rps12/7 PCR fragment was treated with SalI and HindIII enzymes and inserted into pTCP101s1 between the SalI and HindIII sites, upstream of the chimeric aadA3 gene. This resulted in a 6998-bp master expression plasmid pTCP101 for tobacco chloroplast transformation. In this plasmid, each element was separated by a unique restriction site: XhoI was between 16S rDNA-trnV and psbA terminator, PstI was between psbA terminator and aadA3, and NcoI was between aadA3 and Prrn. Outside of the 16S rDNA-trnV and rps12/7 sequences, there were KpnI and HindIII sites, respectively. These sites enabled easy replacement of each element. Upstream of Prrn, a polylinker region containing unique SalI, SmaI, NotI, and AvrII sites was added. These sites allowed introduction of other transgene constructs. The pTCP101 plasmid is shown in FIG. 2.

To make a chloroplast expression plasmid for transformation of the cpNTrbcL chimeric gene, the pTCP10 plasmid was treated with NotI and SalI enzymes. The DNA fragment containing the psbA Pro::cpNTrbcL::rps16 Ter chimeric gene was purified from pTCP10 using QIAquick Gel Extraction Kit (Qiagen). It was inserted into pTCP101 in the polylinker sequence between NotI and SalI sites and the resulting expression plasmid was named pTCP102 (FIG. 3). This plasmid had tobacco chloroplast genomic sequences rps12/7 and 16SrDNA-trnV flanking the chimeric transgenes. Through homologous recombination, the transgenes bounded by these sequences can be introduced into the tobacco chloroplast genome. This plasmid had two expression cassettes oriented in opposite directions, away from each other. One cassette was NTpsbA Pro::cpNTrbcL::NTrps16 Ter, for expression of tobacco RUBISCO LSU with a C-terminal 6-His tag. The other one was NTPrrn::aadA3::NTpsbA Ter, to allow selection of transformants using spectinomycin. The next step was to transform the transgenes created above into the rbcL-knockout tobacco chloroplast.

### Chloroplast Transformation of Nicotiana Tabacum

The rbcL Knockout Line of Nicotiana tabacum

The rbcL-knockout (rbcL-KO) tobacco plant (see detailed description above) was chosen as the recipient of the cpNTrbcL transgene.

### Transformation and Regeneration

The rbcL-KO line was grown under sterile conditions using ½ strength MS medium (Sigma-Aldrich) contained in standard Magenta Tissue Culture Boxes. Sterile leaves (3-7 cm in length) were excised and one leaf was placed abaxial side up on the SAFC modified MS medium (SAFC Biosciences, Lenexa, Kans.) before bombardment. Leaves were bombarded with pTCP102 DNA-coated gold particles (average diameter 0.6 μm) using the Bio-Rad PDS-1000/He Particle Bombardment System (Bio-Rad, Hercules, Calif.) using manufacturer's directions. The particles (25 mg in 100 μl of distilled H₂O) were coated by sequential addition of DNA (100 μg in 100 μl of H₂O, 100 μl of 2.5M CaCl₂, and 160 μl of 1.0 M spermidine free base). Following these additions, the particles were rinsed twice with absolute ethanol and then resuspended in 50 μl of absolute ethanol. The DNA coated particles (a 5.0 μl aliquot containing 2.5 mg of particles coated with 10 μg of DNA) was then placed on the flying disk. The tissue was placed about 3 inches from the stopping screen and a burst pressure of 1100 psi was used. After bombardment, the leaf was placed on the agarose-solidified and hormone-containing T867 medium (Phyto Technology, Lenexa, Kans.) with the bottom surface of the leaf in contact with the medium. Two days after incubation on this medium, the leaves were cut into 1-2 cm size squares and placed onto fresh T867 medium containing 500 mg/l spectinomycin. The tissue was subsequently transferred to fresh spectinomycin-containing medium every 10 days. Spectinomycin-resistant calli and shoots were recovered after about 8 weeks of incubation on the selection medium. Shoots were generated from the resistant tissue on the SAFC modified MS medium containing 500 mg/l spectinomycin. All media described above were supplemented with 8 g/l agar and 30 g/l sucrose to support growth. Following two months growth on the MS medium green transformant plants such as 81021, 81311, and 81321 and pale transformant plants such as 81051 were obtained. The next step was characterization of the transplastomic tobacco plants.

### Example 3

### Characterization of Transplastomic Tobacco

Transgene Insertion into the Chloroplast Genome

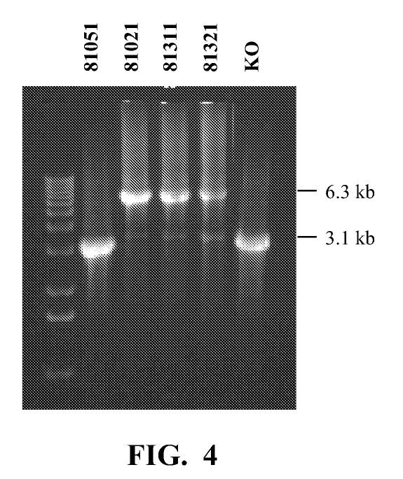

Transformants 81021, 81051, 81311, and 81321 were derived from transformation of the rbcL-KO line with pTCP102. Total DNA was isolated from leaves of two-month old transformants using DNeasy Plant Mini Kit, following the manufacturer's instructions (Qiagen, Valencia, Calif.). DNA concentration was determined by using Nanodrop ND-1000 (Nanodrop technologies, Montchanin, Del.). To detect DNA insertion, 2 ng DNA was used in 50-μL PCR reactions which had been assembled with an Expand Long Template PCR System Kit (Roche Diagnostics, Indianapolis, Ind.). Reactions included two primers. Primer rbc146 (SEQ ID NO: 15) was a forward primer complementary to a sequence 127 bp upstream of the 16SrDNA/trnV recombination site in the tobacco chloroplast genome, while primer rbc147 (SEQ ID NO: 16) was a reverse primer complementary to a sequence 151 bp downstream of the rps12/7 recombination site in the same genome. Reactions were incubated for 2 min at 94° C., followed by 35 cycles of 10 sec at 94° C., 30 sec at 56° C., and 5 min at 68° C. In the last 25 cycles, elongation at 68° C. was 20 sec longer for each successive cycle. Picture of an EtBr-agarose gel of 5.0 μl of each PCR reaction is shown in FIG. 4. It can be seen that a 6.3 kb fragment was amplified from the chloroplast genomic DNA of the 81021, 81311, and 81321 plants, as would be expected following insertion of the two transgenes in these plants. Similar to the rbcL-KO plant, a shorter 3.1 kb fragment was amplified from the chloroplast genomic DNA of the 81051 plant. There appears therefore to be no transgene insertion in this plant which might explain its phenotypic similarity to the rbcL-KO plant. To further confirm transgene insertion, the 6.3 kb fragments were excised from the gel and purified using the QIAquick Gel Extraction Kit (Qiagen). The fragments were sequenced using primers rbc146 (SEQ ID NO: 15) and rbc147 (SEQ ID NO: 16) to examine the sequences at junctions between the transgene and chloroplast DNA. Results confirmed that the transgenes had been precisely inserted into the tobacco chloroplast genome, between the 16S rDNA/trnV and rps12/7 loci, through homologous recombination mediated by the homologous targeting sequences.

Expression of cpNTrbcL Transcript

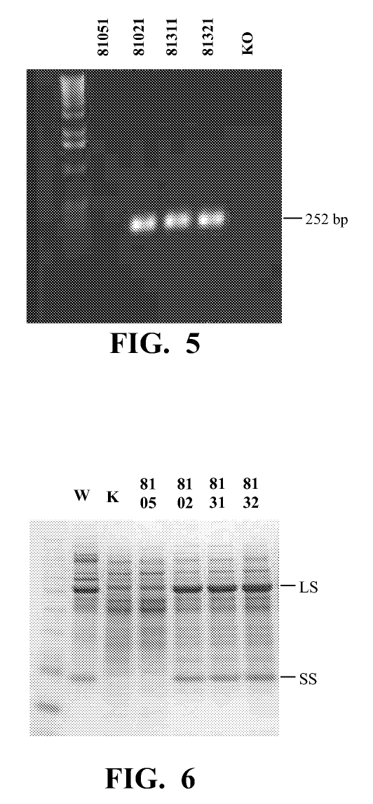

Total RNA was isolated from leaves of two-month old transformants 81021, 81051, 81311, and 81321 using the RNeasy Plant Mini Kit, following the kit's instructions (Qiagen). The RNA concentration was determined using the Nanodrop ND-1000 assay and expression of the rbcL transcript was examined by an RT-PCR assay. First, the QuantiTech Reverse Transcription Kit (Qiagen) was used to make cDNA from 2 μg isolated RNA. The reverse transcription reaction was performed at 42° C. for 30 min. Then, 1.0 μl of the reverse transcription reaction was subjected to a PCR reaction using HotStartTaq PCR Kit (Qiagen) as recommended by the manufacturer. Primer rbc90 (SEQ ID NO: 17) and primer ATrbcL (SEQ ID NO: 18) were used in the reaction to amplify a 252-bp fragment from the cpNTrbcL transgene. The PCR reaction was performed for 15 min at 95° C., followed by 25 cycles of 30 sec at 94° C., 30 sec at 56° C., and 30 sec at 72° C. Analysis of a 5 μl aliquot of this reaction on an EtBr-agarose gel confirmed expression of cpNTrbcL in the 81021, 81311, and 81321 plants, however, expression was absent in the 81051 and rbcL-KO plants (FIG. 5).

In addition, qPCR analysis of cDNA was performed. For this analysis, primer Trans-Tob-rbcL-1337F (SEQ ID NO: 19) and primer Trans-Tob-rbcL-1449R (SEQ ID NO: 20) were used to detect a cpNTrbcL transcript in the transplastomic plants. Primer Endo-Tob-rbcL-1289F (SEQ ID NO: 21) and primer Endo-Tob-rbcL-1362R (SEQ ID NO: 22) were used to detect the presence of the rbcL transcript in wild type plants. One 20 μl quantitative real-time PCR reaction included 10 μl SYBR Green Mix (ABI, Foster City, Calif.), 1.0 μM of each primer, and 1.0 ng cDNA. The real-time PCR was incubated for 10 min at 95° C., followed by 40 cycles of 15 sec at 95° C. and 1.0 min at 60° C. using ABI 7900 SDS system. Relative quantification of the transcript was determined using the ΔΔCt method provided by ABI Co. All data were normalized to tobacco 18S rRNA, which was quantified in a real-time PCR using primers tobacco 18S-1515F (SEQ ID NO: 23) and tobacco 18S-1580R (SEQ ID NO: 24). The relative quantification of cpNTrbcL transcript in 81051, 81021, 81311, and 81321 plants is shown in Table 4. The amount of cpNTrbcL transcript in the 81021, 81311, and 81321 plants was approximately 2 times higher than the rbcL transcript in the wild type tobacco. In the 81051 plant, no cpNTrbcL transcript was present. It should be noted that wild type and transformants were grown under different conditions. For example, the transformants were grown in much weaker illumination and were younger than the wild type tobacco. The two-fold difference of relative transcript concentration between them described above might be caused by these different growth conditions, rather than an inherent difference in the structure of the genes.

TABLE 4
Real-time PCR to determine relative quantification of cpNTrbcL
transcript in transformants of pTCP102. [Triplicate assays
were performed and the average reported].

| | Relative | |
|---|---|---|
| Plant | Quantification | Std Dev |
| Wild Type | 1.00 | ±0.06 |
| 81051 | 0.00 | ±0.00 |
| 81021 | 1.94 | ±0.04 |
| 81311 | 2.71 | ±0.13 |
| 81321 | 2.65 | ±0.07 |

Accumulation of the cpNTrbcL Protein Product

A soluble extract was prepared by grinding 150 mg leaves of two-month old transformants in ice-cold 200 μl leaf extraction buffer using a FastPrep (MP Biomedicals, Solon. Ohio). The buffer contained 50 mM Tris-HCl at pH 8.0, 50 mM NaCl, 0.1 mM EDTA, 2 mM DTT, 5 mM MgCl₂, 5% glycerol, and 1% protease inhibitor cocktail for plants (Sigma). Cell debris was removed by centrifugation at 10,000×g at 4° C. for 15 min. Protein concentration in the supernatant was determined using the Coomassie Plus Protein Assay Reagent (Pierce, Rockford, Ill.). To measure accumulation of the cpNTrbcL product, the 6-His tagged tobacco RUBISCO LSU, leaf protein extract containing 6 μg TSP was subjected to SDS-PAGE on a 4%-12% Novex Bis-Tris Gel (Invitrogen, Carlsbad, Calif.). Sample pre-treatment and electrophoresis were conducted using NuPAGE reagents and following the NuPAGE Technical Guide (Invitrogen) followed by staining with GelCode Blue Stain Reagent (Pierce, Co). These analyses indicated that while plants 81021, 81311, and 81321 had accumulated substantial amounts (comparable to levels in the wild type tobacco) of RUBISCO LSU, plants 81051 and rbcL-KO did not contain any detectable levels (FIG. 6). Similar to the wild type tobacco, transformants showing high levels of RUBISCO LSU also accumulated high levels of RUBISCO SSU, suggesting that assembly of the RUBISCO holoenzyme stabilized the SSU.

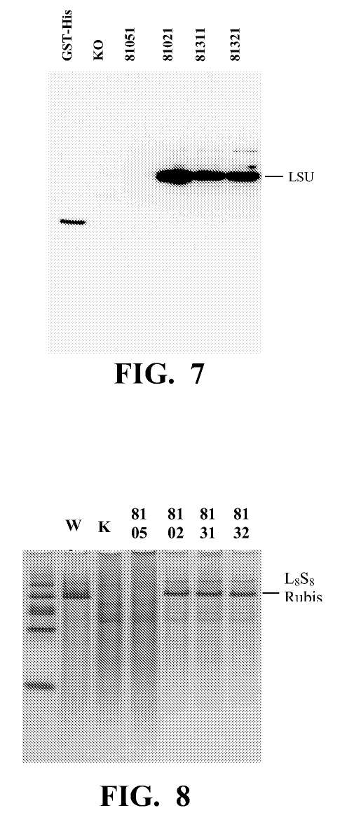

To further confirm that the detected LSU was due to the presence of cpNTrbcL and to quantify its accumulation, protein extracts were resolved on a NuPAGE SDS gel and then transferred to a nitrocellulose membrane (Invitrogen) using a Pharmacia-LKB 2117 multiphor II (Pharmacia Biotech, Piscataway, N.J.), sandwiched by 2 layers of Whatman #1 filter paper on both sides. The gel and filter were wetted with semi-dry western transfer buffer (40 mM glycine, 50 mM Tris, 1.0 mM SDS, and 20% methanol). Transfer was carried out at 0.8 mA/cm² for 1.5 hr. The protein bound to the membrane was probed with 1,000× diluted Anti-His (C-term)-HRP Antibody (Invitrogen) and detected with SuperSignal West Pico Chemiluminescent Substrate Solution (Pierce Co.) in a standard western blot assay. Analysis using a Lumi-Imager (Roche Diagnostics), showed that the LSU that accumulated in plants 81021, 81311, and 81321 was indeed a product of the cpNTrbcL transgene since it had a 6-His tag, while plants 81051 and rbcL-KO did not show His-tagged LSU accumulation even in the highly sensitive western blot assay (FIG. 7). In this assay, a purified GST-6His fusion protein was used as the control. By measuring signal intensities of GST-6His and LSU-6His, accumulation levels of the cpNTrbcL product were calculated as 54% of TSP in plant 81021, 50% of TSP in plant 81311, and 49% of TSP in plant 81321. In wild type (not shown) and rbcL-KO plants and in the 81051 transgenic plant there was no signal detected using this antibody.

### Assembly of RUBISCO Holoenzyme

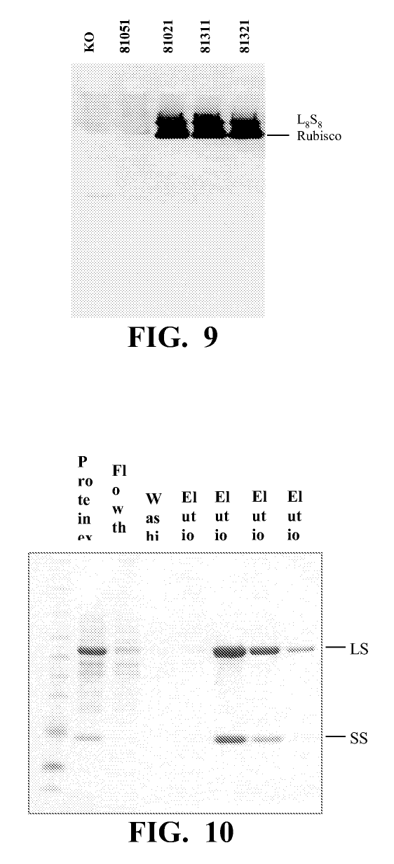

To confirm assembly in the transformants of transgenic RUBISCO LSU and endogenous SSU into the RUBISCO holoenzyme, described above, leaf protein extract containing 6 μg TSP was analyzed using a 10% Novex Tris-Glycine Native-PAGE Gel (Invitrogen, Carlsbad, Calif.). Sample pre-treatment and electrophoresis were performed using Invitrogen native electrophoresis reagents as recommended by the manufacturer (Invitrogen) and the gel was stained with Pierce's GelCode Blue Stain Reagent (FIG. 8). Results showed that plants 81021, 81311, and 81321 had significant accumulation of a protein complex with the same size as the 550 kD L₈S₈ RUBISCO holoenzyme of wild type tobacco, while plants 81051 and rbcL-KO did not show accumulation of this complex. Proteins separated on the Novex Native gel were transferred to a nitrocellulose membrane and analyzed using a western blot assay with Anti-His (C-term)-HRP Antibody (Invitrogen) and results were recorded by a Lumi-Imager as described above (FIG. 9). The results showed that the 550 kD complex accumulated in plants 81021, 81311, and 81321 contained the LSU-6His tag.

RUBISCO complex assembly in the transformant was further studied by purifying the complex from plant 81021. For this purpose, 1.0 g leaf tissue of this plant was ground in liquid nitrogen, mixed with 2.5 ml protein extraction buffer (0.1 M NaEPPS pH8.0, 2.5 mM MgCl₂, 0.1 mM EDTA, 10 mM NaHCO₃, 10 mM NaHSO₃, 10 mM 2-mercaptoethanol), then micro-centrifuged twice at 14,000 rpm for 15 min at 4° C. to remove cell debris. The concentration of soluble protein was determined using Coomassie Plus Protein Assay Reagent (Pierce). The protein extract was mixed with 0.25 ml Ni-NTA resin (Invitrogen) for 2 hr with gentle agitation to bind LSU-6His to the resin. The mixture was loaded onto a column to collect the flow through fraction and then washed with 8 ml protein extraction buffer. Finally, proteins on the column were eluted 4 times with 0.4 ml elution buffer (protein extraction buffer with 0.3 M imidazole and 10 mM EDTA) and eluted fractions were collected separately. To examine protein profiles during the course of purification, 1.0 μl crude protein extract and 5.5 μl of each collected fraction were separated by electrophoresis using a NuPAGE Novex Bis-Tris Gel and stained as described above. Results showed that not only LSU-6His but also endogenous SSU had been purified (FIG. 10). Since the endogenous SSU had no 6-His tag, these results indicated that endogenous SSU interacted with the transgenic LSU-6His and formed a hybrid RUBISCO holoenzyme.

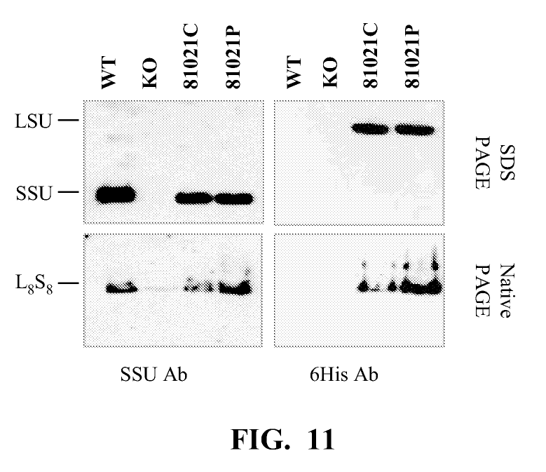

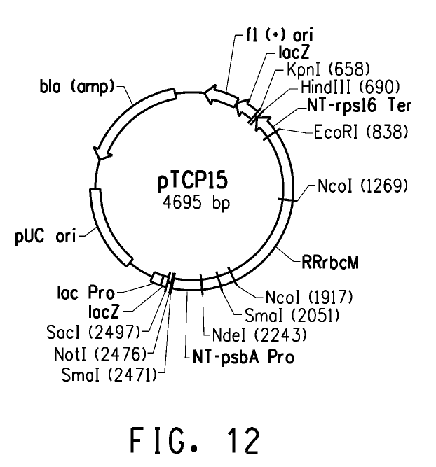

As a further test of the formation of the complex, crude protein extract (loading fraction, 0.6 μg protein) and purified protein (eluted fraction, 0.3 μg protein) were analyzed by both SDS-PAGE and Native-PAGE western blot as described above. Wild type tobacco extract containing 2.5 μg protein and rbcL-KO tobacco extract containing 6 μg protein were used as positive and negative controls, respectively. The western blots were probed by 1,000× diluted Anti-His (C-term)-HRP Antibody (Invitrogen) or by 2,000× diluted Anti-NTrbcS Antibody and then by 10,000× diluted Anti-Rabbit IgG-HRP (Jackson Immuno Research, West Grove, Pa.). Results using the Anti-His antibody confirmed that the larger purified protein had a 6-His tag and thus was an LSU-6His (FIG. 11, upper right panel). The LSU-6His protein was located in a 550 kD RUBISCO complex in both crude protein extract and purified protein (FIG. 11, lower right panel). Since neither the wild type nor the rbcL-KO plants hosted a cpNTrbcL transgene, no LSU-6His was detected in protein extracts of these plants. Results using the Anti-SSU antibody demonstrated that the smaller purified protein was an endogenous tobacco RUBISCO SSU (FIG. 11, upper left panel). This protein was also located in a 550 kD RUBISCO complex in both the crude protein extract and the purified protein (FIG. 11, lower left panel) which was detected because the SSU had accumulated in the wild type tobacco. These experiments confirmed that, in transplastomic tobacco plants, LSU-6His had formed an L₈S₈ RUBISCO complex by interacting with the endogenous tobacco RUBISCO SSU.

### Activity of the RUBISCO Complex in the Transformants

To demonstrate activity of the RUBISCO holoenzyme that consisted of the endogenous RUBISCO SSU and the transgenic RUBISCO LSU, leaf protein extracts of plants 81051, 81021, 81311, and 81321, as well as the purified RUBISCO complex from plant 81021 were dialyzed overnight against a solution consisting of 0.1 M NaEPPS, pH8.0, 2.5 mM MgCl₂, 0.1 mM EDTA, 10 mM NaHCO₃, 10 mM NaHSO₃, and 10 mM 2-mercaptoethanol. The RUBISCO activity in these samples was measured using the ribulose-1,5-bisphosphate (RuBP) dependent ¹⁴CO₂ fixation assay as described above These data (Table 5) confirmed that the RUBISCO complex that consisted of endogenous RUBISCO SSU and transgenic RUBISCO LSU possessed enzymatic activity. Interference of the N-terminal 6-His tag in the transgenic RUBISCO LSU could account for the observed lower RUBISCO activity in the transgenic plants compared to the wild type. As expected, no RUBISCO activity was observed in either the 81051 or the rbcL-KO plants (Table 5). Detection of higher levels (700 mU/mg) of RUBISCO activity in purified samples of 81021 confirmed proper reconstitution of the RUBISCO complex in the transgenic plant.

TABLE 5
RUBISCO activity in leaf protein extracts and purified
RUBISCO fractions

| | RUBISCO Activity |
|---|---|
| Sample | (mU/mg) |
| Protein extract of WT tobacco | 410 |
| Protein extract of rbcL-KO tobacco | 0 |
| Protein extract of 81051 tobacco | 0 |
| Protein extract of 81021 tobacco | 247 |
| Protein extract of 81311 tobacco | 242 |
| Protein extract of 81321 tobacco | 260 |
| Purified RUBISCO from 81021 | 700 |
| tobacco | |

### Example 4

### Transformation and Expression of the Procaryotic R. Rubrum RUBISCO Transgene in Tobacco

In the above Examples, functional expression of the tobacco RUBISCO gene in tobacco chloroplasts, using plasmid pTCP102 (cpNTrbcL), was demonstrated. To further demonstrate application of this novel expression vector, the RUBISCO gene from the bacterium, R. rubrum (RRrbcM gene) was inserted into the same vector and transformed into the tobacco chloroplast. Expression of the prokaryotic RUBISCO in the tobacco plant was characterized in a similar approach outlined above for pTCP102 transformants, and is described briefly below.

### RRrbcM Transgene and Expression Vector

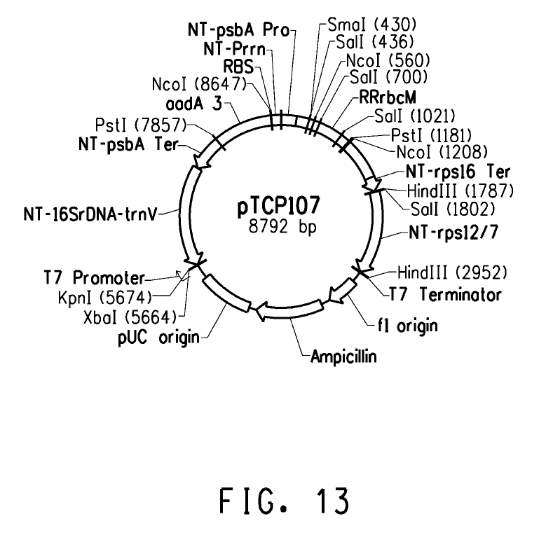

The RUBISCO LSU encoded by the RRrbcM gene of R. rubrum has 30.5% identity to tobacco LSU and forms an L₂ RUBISCO complex in R. rubrum. This gene was amplified from DuPont plasmid stock 3B1 containing a wild type RRrbcM in a PCR reaction, using the Pfu ultra enzyme as described above. Primers rbc154 (SEQ ID NO: 25) and rbc155 (SEQ ID NO: 26) were used in this reaction and the resulting 1401 bp RRrbcM gene encoded a 466-aa RRrbcLSU with a stop codon. It kept an existing NdeI site at the start codon and had an additional EcoRI site after the stop codon. A chimeric RRrbcM transgene construct was formed using pTCP11 which was derived from pTCP10 by replacing NTrbcL of N. tabacum between NcoI and EcoRI sites with AErbcL of Amaranthus edulis. First, two nucleotides A and T were added into pTCP11 at the 5′ end of AErbcL to destroy an NcoI site and create an Ndel site by using Quikchange Site-Direct Mutagenesis Kit (Stratagene). Primers rbc141 (SEQ ID NO: 27) and rbc142 (SEQ ID NO: 28) were used in the mutagenesis reactions and the manufacturer's directions were followed. The amplified RRrbcM was then treated with NdeI and EcoRI, purified using QIAquick PCR Purification Kit (Qiagen), and inserted into the mutated pTCP11 between NdeI and EcoRI site to substitute for AErbcL. The resulting plasmid was pTCP15, containing a chimeric structure of psbA Pro::RRrbcM::rps16 Ter. A map of pTCP15 is shown in FIG. 12 and the DNA sequence of RRrbcM (SEQ ID NO: 35) and its amino acid sequence (SEQ ID NO: 36) are provided. To make a chloroplast expression plasmid for transformation of the RRrbcM transgene, the pTCP15 plasmid was treated with NotI and XhoI enzymes. A chimeric structure of psbA Pro::RRrbcM::rps16 Ter was isolated from pTCP15, purified using a QIAquick Gel Extraction Kit (Qiagen) and inserted into the master plasmid pTCP101 in the polylinker sequence between NotI and SalI sites. The resulting expression plasmid (pTCP107) had two expression cassettes oriented in opposite directions: One cassette was NTpsbA Pro::RRrbcM::NTrps16 Ter, which produced RRrbcLSU and the other one was NTPrrn::aadA3::NTpsbA Ter, which allowed selection of transformants using spectinomycin as the marker (FIG. 13).

### Tobacco Transformation and Transgene Insertion

Plasmid pTCP107, containing the R. rubrum RUBISCO gene, was transformed into the rbcL-KO line of tobacco and transformants were selected and regenerated using standard procedures. The transplastomic plants were then grown on the SAFC modified MS agar medium containing 500 mg/l spectinomycin and 30 g/l sucrose. A two-month old transformant (designated 1074) of this construct was a green plant, appearing similar to the wild type tobacco when grown in shoot culture on a sucrose-containing medium. This tissue-cultured plant was used for molecular characterization described below. This plant was also grown in soil. However, due to poor kinetic properties of the R. rubrum RUBISCO, this plant could only survive and grow to maturation when 3,000 ppm CO₂ was supplemented.

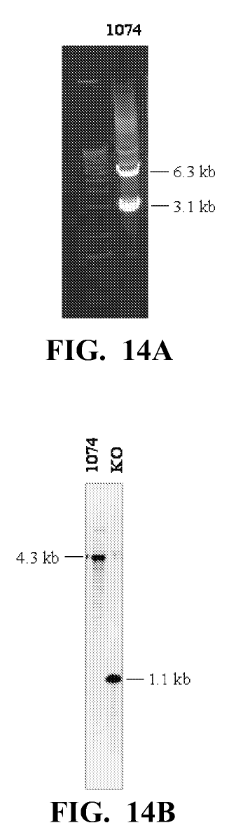

To examine the transgene insertion in the chloroplast genome, total DNA was isolated from the leaf tissue of plant 1074 and used in a PCR reaction that was assembled with an Expand Long Template PCR System Kit (Roche Diagnostics). The reaction included primers rbc146 (SEQ ID NO: 15) and rbc147 (SEQ ID NO: 16). As described above, rbc146 was a forward primer complementary to a sequence 127 bp upstream of the 16SrDNA/trnV recombination site in the tobacco chloroplast genome, while rbc147 was a reverse primer complementary to a sequence 151 bp downstream of the rps12/7 recombination site in the same genome. This reaction amplified two products with equal intensity from the DNA preparation (FIG. 14A). The presence of a 6.3 kb fragment was indicative of the correct transgene insertion in this plant. The presence of a shorter 3.1 kb fragment in these samples suggested the potential of the presence of some non-transformed plastomes in this plant.

Since PCR reactions preferentially amplify smaller fragments, the amounts of the 2 fragments may not accurately reflect the ratio of the transformed and non-transformed plastomes. Therefore, total DNA from plants 1074 and rbcL-KO, as a control, were used in a southern blot assay for further investigation. One pg of each DNA sample was digested with BspEI and BstEII, separated by agarose gel electrophoresis, and blotted to a nylon membrane using standard procedures. A DIG-labeled DNA probe was synthesized in a PCR-based reaction using a PCR DIG Probe Synthesis Kit (Roche Diagnostics). In this reaction, primers rbc211 (SEQ IDNO: 29) and rbc212 (SEQ ID NO: 30) were used to produce a 628 bp fragment that matched a segment of the 16S rDNA-trnV operon in the tobacco chloroplast genome and a part of 16S rDNA-trnV recombination fragment in pTCP107. The DIG-labeled probe was hybridized to the DNA blot and detected in a luminescent reaction using the DIG High Prime DNA Label/Detection Kit (Roche Diagnostics) following manufacturer's instructions (FIG. 14B). Compared to the 1.1 kb fragment in the DNA sample of the rbcL-KO line, only a larger 4.3 kb fragment was detected from the DNA sample of plant 1074, which demonstrated that all or at least the great majority of plastomes had been transformed in this plant. If there were non-transformed plastomes, their numbers were too small to be detected by the Southern blot assay.

To confirm the accuracy of transgene insertion, the 6.3 kb fragment in the PCR assay from plant 1074 was excised from the gel and purified using the QIAquick Gel Extraction Kit (Qiagen). The fragment was sequenced using primers rbc146 and rbc147 to examine the sequences at the junctions between the transgene and the chloroplast DNA. The results confirmed that the 16S rDNA/trnV and rps12/7 sequences flanking the transgenes in pTCP107 had seamlessly integrated into the same sequences located in the tobacco chloroplast genome through homologous recombination. Further sequence analysis indicated that the transgenes had been accurately inserted between the 16S rDNA/trnV and the rps12/7 sections of the tobacco chloroplast genome.

### Characterization of the RRrbc LSU Transgene Expression

Characterization of the 1074 transformant focused on the expression, complex assembly and activity of the prokaryotic RRrbc LSU. Technical details were similar to those described above in detail for the eukaryotic pTCP102 transformants. A TSP extract was prepared from the leaf tissue of the tissue cultured plant 1074 and the protein concentration was determined.

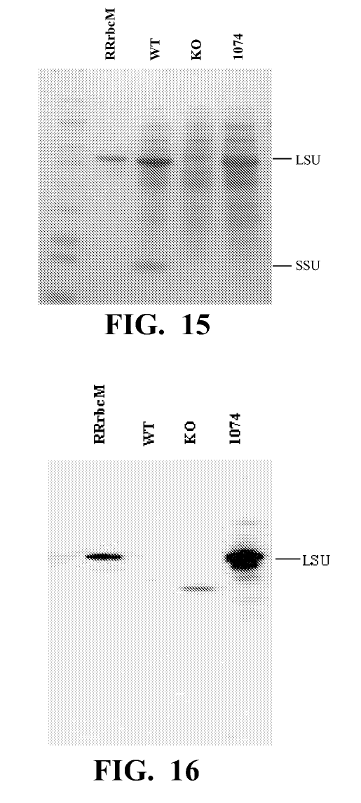

To measure accumulation of RRrbc LSU, the product of RRrbcM, the crude leaf extract was analyzed by SDS-PAGE analysis using a 4%-12% NuPAGE Novex Bis-Tris Gel (Invitrogen). Crude leaf extracts from wild type and rbcL-KO tobacco and purified R. rubrum RUBISCO were used as controls and the gel was stained with GelCode Blue Stain Reagent (Pierce) (FIG. 15). These analyses showed that the levels of 55-kD RUBISCO LSU protein accumulated in plant 1074 were comparable to those found in wild type tobacco and the protein's molecular mass was close to both R. rubrum and tobacco RUBISCO LSU proteins. However, the plant did not accumulate the tobacco RUBISCO SSU, indicating that this transgenic 55-kD RUBISCO-like protein did not form a stable complex with the tobacco SSU. In a western blot analysis, 50 ng purified R. rubrum RUBISCO and 0.5 μg protein from plant 1074 were loaded on the SDS-PAGE gel, the separated proteins were transferred to a nitrocellulose membrane and probed with 5,000× diluted Anti-Rubrum RUBISCO Antibody as the first antibody and 10,000× diluted Anti-Rabbit IgG-HRP (Jackson Immuno Research) as the secondary antibody. Accumulation of R. rubrum RUBISCO was detected in a chemiluminescent reaction and recorded using a Lumi-Imager. Results shown in FIG. 16 confirmed that the 55-kD RUBISCO-like protein accumulated in plant 1074 was indeed the R. rubrum RUBISCO protein, since in the same assay, only the purified R. rubrum RUBISCO and not the tobacco RUBISCO LSU was detected. By measuring signal intensities of the purified and expressed R. rubrum RUBISCO proteins, the accumulation level of RRrbcM product was estimated to be 41% of the TSP in plant 1074.

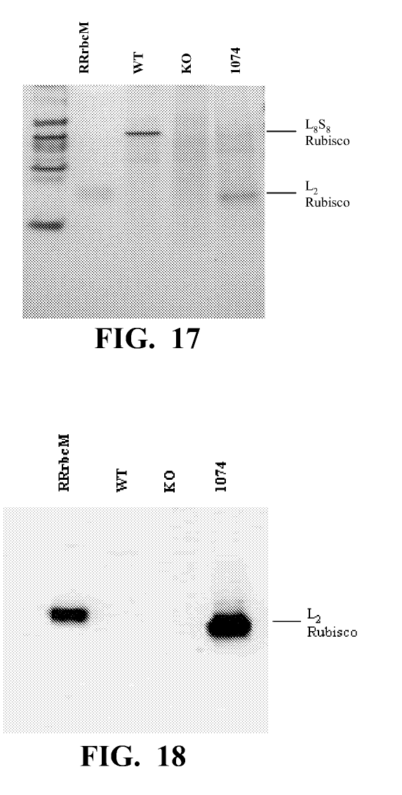

To study assembly of the R. rubrum RUBISCO protein in the plant, these protein samples were analyzed on two 10% Novex Tris-Glycine Native-PAGE gels (Invitrogen). After electrophoresis, one gel was stained with GelCode Blue Stain Reagent and the other one was examined by immunoblot, as described above. The stained gel visualized the L₂ complex of the purified R. rubrum RUBISCO and the L₈S₈ complex of the wild-type tobacco RUBISCO and the RUBISCO formed in plant 1074 had the same mobility as the purified R. rubrum L₂ complex (FIG. 17). Immunoblot analysis demonstrated that the L₂-like complex indeed consisted of the expressed R. rubrum RUBISCO protein, since it was recognized by anti-R. rubrum RUBISCO antibody (FIG. 18).

To demonstrate the activity of the plant-expressed R. rubrum RUBISCO, the leaf protein extract of plant 1074 was used in a RUBISCO activity assay as described above. The protein extract made from E. coli strain pRR2119 (Somerville and Somerville, Mol. Gen. Genet., 193, 214-219, 1984), overexpressing the RRrbcM gene, was used as a positive control to show that the assay was able to detect activity of a functional R. rubrum RUBISCO complex. An extract of the rbcL-KO tobacco was used as a negative control. Experimental results are summarized in Table 6, showing the presence of 160 mU/mg protein of RUBISCO activity in plant 1074 while the rbcL-KO tobacco did not have any activity.

TABLE 6
R. rubrum RUBISCO ACTIVITY IN TOBACCO AND E. coli

| Sample (Protein extract) | RUBISCO Activity (mU/mg) |
|---|---|
| E. coli pRR2119 | 640 |
| (overexpressing RRrbcM) | |
| rbcL-KO tobacco | 0 |
| 1074 tobacco | 160 |

What is claimed is:

1. A plant cell comprising a chloroplast genome having inserted therein a heterologous genetic construct encoding a RUBISCO enzyme, wherein the genetic construct is inserted at a non-RUBISCO locus in the genome and wherein the RUBISCO enzyme is selected from the group consisting of: a RUBISCO large subunit and a RUBISCO small subunit.

2. The plant cell of claim 1 wherein the chloroplast insertion site locus is within the inverted repeat regions of the chloroplast genome.

3. The plant cell of claim 2 wherein the chloroplast insertion site locus is between the trnV and rps7 genes in the inverted repeat regions of the chloroplast genome.

4. The plant cell of claim 1 wherein the RUBISCO enzyme is derived from tobacco or Rhodospirillum.

5. The plant cell of claim 1 wherein the plant cell is derived from a C3 plant.

6. The Plant cell of claim 5 wherein the C3 plant is selected from the group consisting of: corn, rice, soybean, tobacco, and canola.

7. The plant cell of claim 1 wherein the RUBISCO enzyme is expressed in soluble form.

8. The plant cell of claim 7 wherein the soluble RUBISCO enzyme comprises at least about 30% of the soluble proteins of the cell.

9. The plant cell of claim 1 wherein the plant cell does not express a native RUBISCO large subunit.

10. A method for the expression of a RUBISCO enzyme in a plant comprising:
a) Providing a plant comprising a chloroplast genome;
b) Providing a vector consisting essentially of the general structure:
HA1-hetero Pro1::M::Ter1 hetero Pro2::RBC::Ter2-HA2
Wherein:
i) hetero Pro1 is a first promoter derived from a non-RUBISCO plant gene;
ii) M genetic construct encoding a selectable marker;
iii) Ter1 is a first terminator;
iv) hetero Pro2 is a second promoter derived from a non-RUBISCO plant gene;
v) RBC is a genetic construct encoding a protein selected from the group consisting of: the small subunit of a RUBISCO enzyme and the large subunit of an RUBISCO enzyme;
vi) Ter2 is a second terminator;
vii) HA1 is a first homology arm having homology to a first section of the inverted repeat region of the chloroplast genome; and
viii) HA2 is a second homology arm having homology to a second section of the inverted repeat region of the chloroplast genome; and
c) transforming the plant of (a) with the vector of (b) wherein the vector inserts in the inverted repeat region of the chloroplast genome and the RBC is a genetic construct which is expressed producing a RUBISCO enzyme in soluble form.

11. The method of claim 10 wherein hetero Pro1 is a promoter derived from a plastid rRNA operon.

12. The method of claim 10 wherein Ter1 is a terminator derived from a plastid psbA gene.

13. The method of claim 10 wherein Ter2 is a terminator derived from the plastid rps16 gene.

14. The method of claim 10 wherein HA1 and HA2 have homology to portions of the inverted repeat region of the chloroplast genome that reside between the trnV and rps7 genes.

15. The method of claim 10 wherein the RUBISCO enzyme comprises at least about 30% of the total cell soluble proteins.
